SEQUENCE LISTING (8 sequences)

SEQ ID NO: 1 - DNA, 564 bp, Pseudomonas aeruginosa
atggaccactatctcgacattcggctgcgacctgacccggagtttcctcccgcccaacttatgagcgtgctgttcggcaaattgcaccaggccctggtagctcaaggcggtgaccgaattggagtgagcttccctgacctggatgagtctaggtcccgactgggtgagagactcagaatccacgcatccgccgacgacctcagagcactgctggcccgcccctggctggagggcctcagagatcacttgcagtttggagagccagccgtcgtgcctcaccctaccccatacaggcaagtgtctagagtccaggccaagagtaaccccgaacggctgcggcggaggttgatgaggcggcacgacctgtccgaagaagaggcacggaaaagaattcccgacaccgttgctagggctcttgatttgcccttcgtcacccttcgatcacagtccaccggacaacatttccgcctgttcattaggcacgggcctctgcaggtcactgccgaagagggcggattcacttgctacgggctgtccaagggagggttcgttccatggttctga

SEQ ID NO: 2 - DNA, 607 bp, Escherichia coli
atgtacctcagtaagatcatcatcgcccgcgcttggtcccgtgacctgtaccaactgcaccaagagctctggcacctcttccccaacaggccagatgccgctagagacttcctgttccacgtggagaagcgtaacacccccgaagggtgccacgtgctgttgcagagtgcccagatgccagtgagtaccgctgttgccactgtcatcaagactaaacaagttgaattccaactgcaagtgggcgtccctctgtatttccgcctcagggccaaccccatcaaaaccatcctggacaaccagaagcggctggatagcaaaggtaatatcaagagatgccgcgtgcctctgatcaaggaggccgagcagatcgcttggctgcaacgcaagctgggtaacgccgcgagagtggaagatgtgcacccaatctccgagcgcccgcagtatttctccggggaggggaagaacggcaaaattcagactgtctgcttcgagggggtgctcactattaacgacgcccctgctctgatcgacctcctgcagcagggcattgggcccgcgaagagcatgggatgcggattgttgagcctggcacccctgtgagctttga

SEQ ID NO: 3 - DNA, 480 bp, Artificial Sequence
  Synthetic
atgatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgatga

SEQ ID NO: 4 - DNA, 738 bp, Artificial Sequence
  Synthetic
atgagccttgccctgtcacttacagccgaccagatggtttccgcgcttctcgacgctgaacctccaattctctattccgaatacgacccaaccaggccgttctccgaggcatctatgatgggtctgctgacaaatctggcagacagggaactggtgcacatgatcaattgggcgaagcgcgtacccggattcgtcgatcttgcactccatgatcaggtgcacttgctggagtgcgcttggatggagatcctcatgatcgggctggtgtggcggagtatggaacaccccggcaagttgctgtttgcgcctaacctcctgttggacaggaaccaggggaaatgtgtggagggcggtgtggaaatctttgacatgctcctcgctacctcaagccggtttaggatgatgaatctgcagggcgaagagttcgtgtgtctcaaatctatcatactgttgaacagcggagtctacaccttcctctccagtactctgaaatctctggaggagaaagatcatatccatcgcgtgctggacaagataaccgacacgttgattcacttgatggccaaagctgggctcactctgcaacaacaacatcagcgactggcacagctgttgctgattttgagccacattcggcacatgtccagcaagagaatggagcacctctatagtatgaagtgcaagaacgtcgtacccctgtcagatctgcttcttgaaatgcttgatgcccaccggtga

SEQ ID NO: 5 - DNA, 2040 bp, Artificial Sequence
  Synthetic
atgagccttgccctgtcacttacagccgaccagatggtttccgcgcttctcgacgctgaacctccaattctctattccgaatacgacccaaccaggccgttctccgaggcatctatgatgggtctgctgacaaatctggcagacagggaactggtgcacatgatcaattgggcgaagcgcgtacccggattcgtcgatcttgcactccatgatcaggtgcacttgctggagtgcgcttggatggagatcctcatgatcgggctggtgtggcggagtatggaacaccccggcaagttgctgtttgcgcctaacctcctgttggacaggaaccaggggaaatgtgtggagggcggtgtggaaatctttgacatgctcctcgctacctcaagccggtttaggatgatgaatctgcagggcgaagagttcgtgtgtctcaaatctatcatactgttgaacagcggagtctacaccttcctctccagtactctgaaatctctggaggagaaagatcatatccatcgcgtgctggacaagataaccgacacgttgattcacttgatggccaaagctgggctcactctgcaacaacaacatcagcgactggcacagctgttgctgattttgagccacattcggcacatgtccagcaagagaatggagcacctctatagtatgaagtgcaagaacgtcgtacccctgtcagatctgcttcttgaaatgcttgatgcccaccggctgatgagccttgccctgtcacttacagccgaccagatggtttccgcgcttctcgacgctgaacctccaattctctattccgaatacgacccaaccaggccgttctccgaggcatctatgatgggtctgctgacaaatctggcagacagggaactggtgcacatgatcaattgggcgaagcgcgtacccggattcgtcgatcttgcactccatgatcaggtgcacttgctggagtgcgcttggatggagatcctcatgatcgggctggtgtggcggagtatggaacaccccggcaagttgctgtttgcgcctaacctcctgttggacaggaaccaggggaaatgtgtggagggcggtgtggaaatctttgacatgctcctcgctacctcaagccggtttaggatgatgaatctgcagggcgaagagttcgtgtgtctcaaatctatcatactgttgaacagcggagtctacaccttcctctccagtactctgaaatctctggaggagaaagatcatatccatcgcgtgctggacaagataaccgacacgttgattcacttgatggccaaagctgggctcactctgcaacaacaacatcagcgactggcacagctgttgctgattttgagccacattcggcacatgtccagcaagagaatggagcacctctatagtatgaagtgcaagaacgtcgtacccctgtcagatctgcttcttgaaatgcttgatgcccaccggctgatggaccactatctcgacattcggctgcgacctgacccggagtttcctcccgcccaacttatgagcgtgctgttcggcaaattgcaccaggccctggtagctcaaggcggtgaccgaattggagtgagcttccctgacctggatgagtctaggtcccgactgggtgagagactcagaatccacgcatccgccgacgacctcagagcactgctggcccgcccctggctggagggcctcagagatcacttgcagtttggagagccagccgtcgtgcctcaccctaccccatacaggcaagtgtctagagtccaggccaagagtaaccccgaacggctgcggcggaggttgatgaggcggcacgacctgtccgaagaagaggcacggaaaagaattcccgacaccgttgctagggctcttgatttgcccttcgtcacccttcgatcacagtccaccggacaacatttccgcctgttcattaggcacgggcctctgcaggtcactgccgaagagggcggattcacttgctacgggctgtccaagggagggttcgttccatggttctga

SEQ ID NO: 6 - DNA, 1077 bp, Artificial Sequence
  Synthetic
atgtacctcagtaagatcatcatcgcccgcgcttggtcccgtgacctgtaccaactgcaccaagagctctggcacctcttccccaacaggccagatgccgctagagacttcctgttccacgtggagaagcgtaacacccccgaagggtgccacgtgctgttgcagagtgcccagatgccagtgagtaccgctgttgccactgtcatcaagactaaacaagttgaattccaactgcaagtgggcgtccctctgtatttccgcctcagggccaaccccatcaaaaccatcctggacaaccagaagcggctggatagcaaaggtaatatcaagagatgccgcgtgcctctgatcaaggaggccgagcagatcgcttggctgcaacgcaagctgggtaacgccgcgagagtggaagatgtgcacccaatctccgagcgcccgcagtatttctccggggaggggaagaacggcaaaattcagactgtctgcttcgagggggtgctcactattaacgacgcccctgctctgatcgacctcctgcagcagggcattgggcccgcgaagagcatgggatgcggattgttgagcctggcacccctgatgatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgatga

SEQ ID NO: 7 - DNA, 12 bp, Artificial Sequence
  Synthetic
cttatgggttga

SEQ ID NO: 8 - DNA, 12 bp, Artificial Sequence
  Synthetic
accatgggttga

(19) United States
(12) Patent Application Publication
(10) Pub. No.: US 2021/0246439 A1
(43) Pub. Date: Aug. 12, 2021

(54) ENGINEERED BI-STABLE TOGGLE SWITCH AND USES THEREOF

(71) Applicant: Massachusetts Institute of Technology, Cambridge, MA (US)

(72) Inventors: Ron Weiss, Newton, MA (US); Breanna E. DiAndreth, Cambridge, MA (US); Noreen Wauford, Somerville, MA (US)

(73) Assignee: Massachusetts Institute of Technology, Cambridge, MA (US)

(21) Appl. No.: 17/122,087

(22) Filed: Dec. 15, 2020

Related U.S. Application Data
(60) Provisional application No. 62/972,807, filed on Feb. 11, 2020.

(51) Int. Cl. C12N 15/10; C12N 15/11; C12N 15/86

(52) U.S. Cl. CPC C12N 15/1024; C12N 2310/12; C12N 15/86; C12N 15/111

(57) ABSTRACT

The present disclosure, at least in part, provides RNA cleavage based engineered bi-stable toggle switch utilizing the Programmable Endonucleolytic Scission-Induced Stability Tuning (PERSIST) platform. Also provided herein, are vectors encoding the engineered bi-stable toggle switch, and uses thereof.

### RELATED APPLICATION

This application claims the benefit under 35 U.S.C. § 119(e) of U.S. provisional application No. 62/972,807 filed Feb. 11, 2020, which is incorporated by reference herein in its entirety.

### FEDERALLY SPONSORED RESEARCH

This invention was made with Government support under Grant No. R01 CA206218 and R01 CA207029 awarded by the National Institutes of Health (NIH). The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 15, 2020, is named M0656.70476US01-SEQ and is 9 kilobytes in size.

### BACKGROUND

An important challenge in engineering synthetic genetic circuits in mammalian systems is epigenetic silencing. Since current genetic circuits often depend entirely on transcriptional control, they are thus susceptible to epigenetic silencing. Few examples of toggle switches have been made to function well in mammalian cells and be resistant to epigenetic silencing. Further, the field of synthetic biology has not produced a mammalian toggle switch that has shown good fold change between high and low states, stability of these states over multiple days, and responsiveness to switching events.

### SUMMARY

The present disclosure, at least in part, relates to an engineered bi-stable toggle switch controllable by RNA cleavage (e.g., RNA cleavage mediated RNA degradation, or RNA cleavage mediated RNA stabilization) using the Programmable Endonucleolytic Scission-Induced Stability Tuning (PERSIST) platform. Such engineered bi-stable toggle switch comprises two expression cassettes capable of activating themselves and repressing each other according to RNA cleavage signals. The engineered bi-stable toggle switch is also capable of maintaining one state long term in response to a signal, and rapidly switching to the other state in response to a different signal. Various designs can be combined with the engineered bi-stable toggle switch to exert control of the switch. The engineered bi-stable toggle switch described herein may be used for diagnostic or therapeutic applications (e.g., long-term delivery of therapeutic molecules to a subject).

In some aspects, the present disclosure provides an engineered bi-stable toggle switch comprising: (i) a first expression cassette comprising, from 5′ to 3′: a first promoter operably linked to a nucleotide sequence encoding a first copy of a first RNA cleavage site, a coding sequence for a first copy of a first RNA cleavage effector, a nucleotide sequence encoding a first copy of a second RNA cleavage site and a nucleotide sequence encoding a plurality of RNA degradation motifs; and (ii) a second expression cassette comprising, from 5′ to 3′: a second promoter operably linked to a nucleotide sequence encoding a second copy of the second RNA cleavage site, a coding sequence for a first copy of a second RNA cleavage effector, a nucleotide sequence encoding a second copy of the first RNA cleavage site, and a nucleotide sequence encoding a plurality of RNA degradation motifs, wherein the first RNA cleavage effector is orthogonal to the second RNA cleavage effector, wherein the first RNA cleavage effector is capable of cleaving the second RNA cleavage site, and wherein the second RNA cleavage effector is capable of cleaving the first RNA cleavage site.

In some embodiments, the first expression cassette further comprises a nucleotide sequence encoding a first transcript stabilization sequence located 3′ of the coding sequence for the first copy of the first RNA cleavage effector; and/or the second expression cassette further comprises a nucleotide sequence encoding a first transcript stabilization sequence located 3′ of the coding sequence for the first copy of the first RNA cleavage effector. In some embodiments, the first transcript stabilization sequence and the second transcript stabilization sequence each is a triplex. In some embodiments, the triplex is a Metastasis Associated Lung Adenocarcinoma Transcript 1 (MALAT1) triplex.

In some embodiments, the first expression cassette further comprises a coding sequence for a second output molecule operably joined to the coding sequence for the first RNA cleavage effector and a first spacer located between the coding sequence of the first RNA cleavage effector and the coding sequence for the second output molecule; and the second expression cassette further comprises a coding sequence for a first output molecule operably joined to the coding sequence for the second RNA cleavage effector and a second spacer located between the coding sequence of the second RNA cleavage effector and the coding sequence for the first output molecule. In some embodiments, the first spacer and the second spacer is a nucleotide sequence encoding an internal ribosomal entry site (IRES) or a 2A peptide.

In some embodiments, the engineered bi-stable toggle switch described herein further comprises: (iii) a third expression cassette comprising a third promoter operably linked to a coding sequence for a first fusion protein, wherein the first fusion protein comprises a second copy of the first RNA cleavage effector fused to a first protein degradation domain; and (iv) a fourth expression cassette comprising a fourth promoter operably linked to a coding sequence for a second fusion protein, wherein the second fusion protein comprises a second copy of the second RNA cleavage effector fused to a second protein degradation domain, wherein the third promoter and the fourth promoter are each constitutive promoters, wherein the first protein degradation domain is capable of binding to a first small molecule, wherein the second protein degradation domain is capable of binding to a second small molecule, and wherein the first small molecule and the second small molecule are different. In some embodiments, the second copy of the first RNA cleavage effector is fused to the first protein degradation domain directly or through a linker; and/or wherein the second copy of the second RNA cleavage effector is fused to the second protein degradation domain directly or through a linker.

In some embodiments, the first fusion protein comprises more than one of the first protein degradation domain; and/or the second fusion protein comprises more than one of the second protein degradation domain.

In some embodiments, the first protein degradation domain is fused to the N-terminus of the first RNA cleavage effector; and/or the second protein degradation domain is fused to the N-terminus of the second RNA cleavage effector.

In some embodiments, the first protein degradation domain and the second protein degradation domain are DDd, DDe, or DDf. In some embodiments, the first protein degradation domain is DDe and the first small molecule is 4-hydroxytamoxifen (4-OHT); and the second protein degradation domain is DDd and the second small molecule is trimethoprim (TMP).

In some embodiments, the first and second copies of the first RNA cleavage site each comprises a first aptamer sequence capable of binding to a first small molecule, and binding of the first small molecule to the first RNA cleavage site is capable of blocking the second RNA cleavage effector from cleaving the first RNA cleavage site, the first and second copies of the second RNA cleavage site each comprises a second aptamer sequence capable of binding to a second small molecule, and binding of the second small molecule to the second RNA cleavage site is capable of blocking the second RNA cleavage effector from cleaving the first RNA cleavage site, and the first small molecule and the second small molecule are different.

In some embodiments, the first expression cassette comprises a nucleotide sequence encoding a first RNA self-cleavage site operably linked to the first promoter, and wherein the nucleotide sequence encoding the first RNA self-cleavage site is located 5′ of the nucleotide sequence encoding the first copy of the first RNA cleavage site; and the second expression cassette comprises a nucleotide sequence encoding a second RNA self-cleavage site operably linked to the second promoter, and wherein the nucleotide sequence encoding the second RNA self-cleavage site is located 5′ of the nucleotide sequence encoding the second copy of the second RNA cleavage site, wherein first RNA self-cleavage site is different from the second RNA self-cleavage site. In some embodiments, the first RNA self-cleavage site and the second RNA self-cleavage site are ribozymes. In some embodiments, the ribozymes are selected from the group consisting of antigenomic hepatitis delta virus (HDV) ribozyme, genomic HDV ribozyme, and sTRSV hammerhead ribozyme (HHR).

In some embodiments, the first RNA self-cleavage site is capable of self-cleaving in response to a first small molecule, the second RNA self-cleavage site is capable of self-cleaving in response to a second small molecule, and the first small molecule and the second small molecule are different.

In some embodiments, the first promoter and the second promoter are constitutive promoters or inducible promoters.

In some embodiments, the first output molecule and the second output molecule are different, and wherein the first output molecule and the second output molecule are selected from the group consisting of: nucleic acids, therapeutic proteins, and detectable proteins.

In some embodiments, the first RNA cleavage effector and the second RNA cleavage effector are CRISPR endoribonucleases (endoRNAses). In some embodiments, the CRISPR endoRNAses are Cas6, Csy4, CasE, Cse3, LwaCas13a, PspCas13b, RanCas13b, PguCas13b, or RfxCas13d.

In some embodiments, the first transcript stabilization sequence and the second transcript stabilization sequence each is a triplex. In some embodiments, the triplex is a Metastasis Associated Lung Adenocarcinoma Transcript 1 (MALAT1) triplex.

In some embodiments, the plurality of RNA degradation motifs are RNA sequences capable of recruiting deadenylation complexes, miRNA target sites, aptamers comprising binding sites for proteins associated with RNA degradation, aptamers comprising binding sites for engineered proteins that cause RNA degradation.

In some aspects, the present disclosure also provides a vector comprising the engineered bi-stable toggle switch described herein. In some embodiments, the vector is a plasmid, a RNA replicon, or a viral vector. In some embodiments, the viral vector is a lentiviral vector.

In some aspects, the present disclosure also provides a cell comprising the engineered bi-stable toggle switch or the vector described herein. In some embodiments, the cell is a mammalian cell. In some embodiments, the mammalian cell is a human induced pluripotent stem cell (hiPSC), a diseased cell, an immune cell, or a recombinant protein producing cell.

In some embodiments, the cell comprises the engineered bi-stable toggle switch in its genome.

In some aspects, the present disclosure also provides a non-human animal comprising the engineered bi-stable toggle switch, the vector, or the cell described herein. In some embodiments, the non-human animal is a mammal.

In some aspects, the present disclosure also provides a composition comprising the engineered bi-stable toggle switch, the vector, or the cell described herein. In some embodiments, the composition further comprises a pharmaceutically acceptable carrier.

In some aspects, the present disclosure also provides a method of switching gene expression between a first output molecule and a second output molecule, the method comprising: administering to a subject in need thereof the engineered bi-stable toggle switch, the vector, or the cell, or the composition described herein. In some aspects, the present disclosure also provides a method of maintaining long-term ON/OFF regulation of output molecule expression, the method comprising: administering to a subject in need thereof the engineered bi-stable toggle switch, the vector, or the cell, or the composition described herein. In some embodiments, the method described herein further comprising administering the subject with the first small molecule or the second small molecule.

### BRIEF DESCRIPTION OF DRAWINGS

FIGS. 1A-1C are diagrams showing RNA-level ON and OFF switch and the incorporation of CRISPR endoRNAses in the RNA based ON and OFF switches. FIG. 1A is a schematic diagram of RNA-based OFF-switch and ON-switch motif designs that are regulated by RNA cleavage. FIG. 1B includes a chart showing CRISPR endoRNases activate the PERSIST-ON motif and repress the PERSIST-OFF motif. FIG. 1C includes a chart showing evaluation of Cas endoRNase pairwise orthogonality.

FIGS. 2A-2C are diagrams showing the configuration and functionality of the engineered bi-stable toggle switch. FIG. 2A is a schematic representation of the engineered bi-stable toggle switch using two endoRNAses that repress each other and activate themselves via the PERSIST system. A hairpin, with an asterisk (*), is cleavable by Csy4, while a hairpin, with a plus sign (+), is cleavable by CasE. Two reporter proteins—mKO2 and eYFP—were used to reflect the behavior of the toggle switch. Csy4 cleavable hairpin was placed 5′ of mKO2, and CasE cleavable hairpin was placed 5′ of eYFP. FIG. 2B shows bistability behavior of the bi-stable toggle switch across a wide range of ratios that result from different cells receiving different copy number of the plasmids due to the transfections. The genetic circuit delivered to each cell essentially performs a weighted random decision to exhibit either the Csy4 high or CasE high state. FIG. 2C shows the switching behavior of the bi-stable toggle switch, as represented by the percentage of cells expressing eYFP or mKO2 on days 1-3, when additional Csy4 (middle panel) or CasE (bottom panel) were added to the cells transfected with the bi-stable toggle switch.

FIGS. 3A-3J are diagrams showing the configuration and functionality of the bi-stable toggle switch with protein-level degradation domains. FIG. 3A is schematic design of fusion proteins with destabilization domains that cause degradation of the protein in the absence of stabilizing ligand. FIG. 3B are charts showing screening of promoter and degradation domain combinations with Csy4 and quantifying effect on the bistable motif in the presence or absence of 4-OHT or TMP. FIG. 3C are charts showing screening combinations of DDd with CasE and quantifying effect on the bistable motif in the presence or absence of TMP. FIGS. 3D-3F are schematic diagrams showing the structure of the bi-stable toggle switch with DDe-DDe-Csy4 and DDd-CasE fusion proteins, and how such toggle switch behaves in response to 4-OHT or TMP. FIG. 3G is a chart showing the behavior of the toggle switch in response to 4-OHT. FIG. 3H is a chart showing the behavior of the toggle switch in response to TMP. FIG. 3I are charts showing that the bi-stable toggle switch is capable of switching state in response to TMP, and maintain the high CasE high state over at least 48 hours. FIG. 3J are charts showing that a 24-hr dose of 4-OHT can maintain the cell in a Csy4-high/CasE-low state (mKO2-high/eYFP low) for 72 hours after the removal of 4-OHT, and 24-hr dose of TMP can maintain the cell in a Csy4-low/CasE-high state (mKO2-low/eYFP-high) for 72 hours after the removal of TMP. Stacked columns adjacent to flow cytometry plots in FIGS. 3B-3C show proportions of cells in the 1) eYFP^{hi}TagBFP^{hi}, 2) eYFP^{hi} TagBFP^{lo}, 3) eYFP^{lo} TagBFP^{hi}, and 4) eYFP^{lo} TagBFP^{hi} subpopulations. Stacked columns in 3H-3I show proportions of cells in the 1) eYFP^{hi}mKO2^{hi}, 2) eYFP^{hi}mKO2^{lo}, 3) eYFP^{lo}mKO2^{hi}, and 4) eYFP^{lo}mKO2^{lo} subpopulations.

FIGS. 4A-4E are diagrams showing the configuration and functionality of the bi-stable toggle switch with small molecule responsive aptamers. FIG. 4A are schematic diagrams showing a CRISPR target hairpin having a small molecule responsive aptamer. Binding and cleavage of the target site is blocked in the presence of the cognate small molecule. FIGS. 4B-4C are charts showing eYFP expression by HEK293FT cells co-transfected with CasE and eYFP containing a hybrid CasE recognition motif-theophylline aptamer in the 5′ UTR without (FIG. 4B) and with (FIG. 4C) theophylline. FIG. 4D is a schematic diagram showing the behavior of the engineered bi-stable toggle switch with small molecule-responsive aptamers in the absence of small molecules (2 and 4), where all of the RNA binding and cleavage events occur. FIG. 4E is a schematic diagram showing the behavior of the engineered bi-stable toggle switches with small molecule-responsive aptamers in the presence of one small molecule (4*) and the absence of another small molecule (2), where Csy4 binding and cleavage events are hindered.

FIGS. 5A-5C are diagrams showing the configuration and functionality of the bi-stable toggle switch with ribozymes. FIG. 5A is a chart showing that ribozymes are capable of activating the PERSIST-ON motif and repressing the PERSIST-OFF motif. FIG. 5B is a schematic configuration of an engineered bi-stable toggle switch having a ribozyme site.

FIG. 5C is a schematic configuration of an engineered bi-stable toggle switch having a ribozyme site capable of responding to a small molecule, showing the toggle switch activity when one small molecule is present (1*) and another small molecule is absent (4).

### DETAILED DESCRIPTION

The present disclosure, at least in part, relates to an engineered bi-stable toggle switch controllable by RNA cleavage (e.g., RNA cleavage mediated RNA degradation, or RNA cleavage mediated RNA stabilization) using the Programmable Endonucleolytic Scission-Induced Stability Tuning (PERSIST) platform. Such an engineered bi-stable toggle switch comprises two expression cassettes capable of activating themselves and repressing each other according to RNA cleavage signals. The engineered bi-stable toggle switch is also capable of maintaining one state long term in response to a signal, and rapidly switching to the other state in response to a different signal. Various designs can be combined with the engineered bi-stable toggle switch to exert control of the switch. The engineered bi-stable toggle switch described herein may be used for diagnostic or therapeutic applications (e.g., long-term delivery of therapeutic molecules to a subject).

### I. Engineered Bi-Stable Toggle Switch

Some aspects of the present disclosure provide an engineered bi-stable toggle switch. An engineered bi-stable toggle switch, as used herein, refers to a set of two expression cassettes designed to have two expression states. The first expression cassette controls the expression of the first gene, and the second expression cassette controls the expression of the second gene. The expression of the first gene (e.g., a first CRISPR endonuclease) further activates the expression of itself, and represses the expression of the second gene (first gene high state). The expression of the second gene (a second CRISPR endonuclease) further activates the expression of itself, and represses the expression of the first gene (second gene high state). In some embodiments, the engineered bi-stable toggle switch can be switched between the first gene high state and second gene high state in response to various switching signals. In some embodiments, the switching between the first gene high state and second gene high state of engineered bi-stable toggle switch can be controlled by additional regulatory elements (e.g., protein degradation domain, small molecule responsive aptamers, or ribozymes).

In some embodiments, the engineered bi-stable toggle switch described herein is based on RNA cleavage induced RNA degradation or stabilization. Such engineered bi-stable toggle switch incorporates the Programmable Endonucleolytic Scission-Induced Stability Tuning (PERSIST) platform into the expression cassettes so as to control, maintain and switch between different states of the toggle switch. In some embodiments, the PERSIST platform includes a RNA level ON switch for RNA stabilization and a RNA level OFF switch for RNA degradation. In some embodiments, the RNA level OFF switch is designed such that an RNA cleavage site is placed 5′ of a gene coding sequence, and subsequent cleavage at the RNA cleavage site leads to RNA degradation and repression of the gene. In some embodiments, the RNA level ON switch is designed such that an RNA cleavage site is placed 3′ of a gene coding sequence, and subsequent cleavage at the RNA cleavage site leads to RNA stabilization and expression of the gene. The RNA level ON and OFF switch based on PERSIST platform has been previously described, e.g., DiAndreth et al, PERSIST: A programmable RNA regulation platform using CRISPR endoRNases, in a bioRxiv preprint first posted online Dec. 16, 2019 (DiAndreth et al. bioRxiv. (2019). doi: 10.1101/2019.12.15.867150), which is incorporated by reference herein in its entirety.

In some embodiments, the engineered bi-stable toggle switch of the present disclosure incorporates both the RNA level ON switch and OFF switch into the configuration by placing different RNA cleavage sites recognizable by two orthogonal RNA cleavage effector upstream and downstream of the coding sequences for the RNA cleavage effectors. In some embodiments, the present disclosure provides an engineered bi-stable toggle switch comprising: (i) a first expression cassette comprising, from 5′ to 3′: a first promoter operably linked to a nucleotide sequence encoding a first copy of a first RNA cleavage site, a coding sequence for a first copy of a first RNA cleavage effector, a nucleotide sequence encoding a first copy of a second RNA cleavage site and a nucleotide sequence encoding a plurality of RNA degradation motifs; and (ii) a second expression cassette comprising, from 5′ to 3′: a second promoter operably linked to a nucleotide sequence encoding a second copy of the second RNA cleavage site, a coding sequence for a first copy of a second RNA cleavage effector, a nucleotide sequence encoding a second copy of the first RNA cleavage site, and a nucleotide sequence encoding a plurality of RNA degradation motifs, wherein the first RNA cleavage effector is orthogonal to the second RNA cleavage effector, wherein the first RNA cleavage effector is capable of cleaving the second RNA cleavage site, and wherein the second RNA cleavage effector is capable of cleaving the first RNA cleavage site.

An RNA cleavage effector, as used herein, refers to a molecule that cleaves the phosphodiester bond between two ribonucleotides, thus resulting two fragments (a 5′ fragment and a 3′ fragment) of an RNA molecule, such as the RNA transcripts produced by the first expression cassette and the second expression cassette. The RNA cleavage effectors of the present disclosure cleave the RNA transcripts in a sequence-specific manner. Exemplary sequence-specific RNA cleavage effectors include, without limitation, endoribonucleases, RNA interference (RNAi) molecules, and ribozymes (e.g., cis-acting ribozyme or trans-acting ribozyme). The RNA cleavage effector of the present disclosure may directly cleave the RNA transcript (e.g., an endoribonuclease or a ribozyme) or indirectly leads to the cleavage of the RNA transcript (e.g., via the recruitment of other factors that carrier out the cleavage). A non-limiting example of an RNA cleavage effector that indirectly cleaves the RNA transcript is an RNAi molecule, which is incorporated in a RNA-induced silencing complex (RISC) that binds and cleaves a target sequence in the RNA transcript.

In some embodiments, the RNA cleavage effector is an endoribonuclease. An “endoribonuclease,” as used herein, refers to a nuclease that cleaves an RNA molecule in a sequence specific manner, e.g., at a recognition site. Sequence-specific endoribonucleases have been described in the art. For example, the Pyrococcus furiosus CRISPR-associated endoribonuclease 6 (Cas6) is found to cleave RNA molecules in a sequence-specific manner (Carte et al., Genes & Dev. 2008. 22: 3489-3496). In another example, endoribonucleases that cleave RNA molecules in a sequence-specific manner are engineered, which recognize an 8-nucleotide (nt) RNA sequence and make a single cleavage in the target (Choudhury et al., Nature Communications 3, 1147 (2012).

In some embodiments, the endoribonuclease belongs to the CRISPR-associated endoribonuclease. In some embodiments, the endoribonuclease belongs to the CRISPR-associated endoribonuclease 6 (Cas6) family. Cas6 nucleases from different bacterial species may be used. Non-limiting examples of Cas6 family nucleases include Cas6, Csy4 (also known as Cas6f), Cse3, and CasE. In some embodiments, the endoribonuclease belongs to the CRISPR-associated endoribonuclease 13 (Cas13) family. Cas13 nucleases from different bacterial species may be used. Non-limiting examples of Cas13 family nucleases include Cas13a, Cas13b, Cas13c, and Cas13d. In some embodiments, the Cas13 family nucleases are waCas13a, PspCas13b, RanCas13b, PguCas13b, and RfxCas13d.

In some embodiments, the first RNA cleavage effector encoded by the first expression cassette is orthogonal to the second RNA cleavage effector encoded by the second expression cassette. “Being orthogonal to each other,” as used herein, means that the two RNA cleavage effectors used in the engineered bi-stable toggle switch have minimal cross-talk with each other's recognition sites. In some embodiments, a pair of orthogonal CRISPR-associated endonucleases is used in the engineered bi-stable toggle switch described herein. In some embodiments, the pair of orthogonal CRISPR-associated endonucleases is CasE and Csy4. The orthogonality of the endonucleases can be evaluated by methods known in the art, and different pairs of endonucleases can be selected for use in the engineered bi-stable toggle switch described herein based on the orthogonality evaluation results.

An exemplary nucleotide sequence
encoding Csy4 is set forth in
SEQ ID NO: 1:
ATGGACCACTATCTCGACATTCGGCTGCGAC
CTGACCCGGAGTTTCCTCCCGCCCAACTTAT
GAGCGTGCTGTTCGGCAAATTGCACCAGGCC
CTGGTAGCTCAAGGCGGTGACCGAATTGGAG
TGAGCTTCCCTGACCTGGATGAGTCTAGGTC
CCGACTGGGTGAGAGACTCAGAATCCACGCA
TCCGCCGACGACCTCAGAGCACTGCTGGCCC
GCCCCTGGCTGGAGGGCCTCAGAGATCACTT
GCAGTTTGGAGAGCCAGCCGTCGTGCCTCAC
CCTACCCCATACAGGCAAGTGTCTAGAGTCC
AGGCCAAGAGTAACCCCGAACGGCTGCGGCG
GAGGTTGATGAGGCGGCACGACCTGTCCGAA
GAAGAGGCACGGAAAAGAATTCCCGACACCG
TTGCTAGGGCTCTTGATTTGCCCTTCGTCAC
CCTTCGATCACAGTCCACCGGACAACATTTC
CGCCTGTTCATTAGGCACGGGCCTCTGCAGG
TCACTGCCGAAGAGGGCGGATTCACTTGCTA
CGGGCTGTCCAAGGGAGGGTTCGTTCCATGG
TTCTGA
An exemplary nucleotide sequence
encoding CasE is set forth in
SEQ ID NO: 2:
ATGTACCTCAGTAAGATCATCATCGCCCGCG
CTTGGTCCCGTGACCTGTACCAACTGCACCA
AGAGCTCTGGCACCTCTTCCCCAACAGGCCA
GATGCCGCTAGAGACTTCCTGTTCCACGTGG
AGAAGCGTAACACCCCCGAAGGGTGCCACGT
GCTGTTGCAGAGTGCCCAGATGCCAGTGAGT
ACCGCTGTTGCCACTGTCATCAAGACTAAAC
AAGTTGAATTCCAACTGCAAGTGGGCGTCCC
TCTGTATTTCCGCCTCAGGGCCAACCCCATC
AAAACCATCCTGGACAACCAGAAGCGGCTGG
ATAGCAAAGGTAATATCAAGAGATGCCGCGT
GCCTCTGATCAAGGAGGCCGAGCAGATCGCT
TGGCTGCAACGCAAGCTGGGTAACGCCGCGA
GAGTGGAAGATGTGCACCCAATCTCCGAGCG
CCCGCAGTATTTCTCCGGGGAGGGGAAGAAC
GGCAAAATTCAGACTGTCTGCTTCGAGGGGG
TGCTCACTATTAACGACGCCCCTGCTCTGAT
CGACCTCCTGCAGCAGGGCATTGGGCCCGCG
AAGAGCATGGGATGCGGATTGTTGAGCCTGG
CACCCCTGTGAGCTTTGA

When an endoribonuclease is used as the RNA cleavage effector, the RNA cleavage site for the RNA cleavage effector in the RNA transcript comprises one or more (e.g., 1, 2, 3, 4, 5, 6, 7, 8, 9, 10 or more) recognition sites for the endoribonuclease. A “RNA cleavage site for an endoribonuclease” refers to a ribonucleotide sequence that is recognized, bound, and cleaved by the endoribonuclease. The recognition site for an endoribonuclease may be 4-20 nucleotides long. For example, the RNA cleavage site may be 4, 5, 6, 7, 8, 9, 10, 11, 12, 13, 14, 15, 16, 17, 18, 19, or 20 nucleotides long. In some embodiments, endoribonuclease cleavage sites that are shorter than 4 ribonucleotides or longer than 20 nucleotides are used.

In some embodiments, the first expression cassette of the engineered bi-stable toggle switch comprises: a first copy of a first RNA cleavage site 5′ of the coding sequence for a first CRISPR− associated endonuclease, and first copy of a second RNA cleavage site 3′ of the coding sequence for the first CRISPR− associated endonuclease; and a second copy of the second RNA cleavage site 5′ of the coding sequence for a second CRISPR-associated endonuclease, and second copy of the first RNA cleavage site 3′ of the coding sequence for the second CRISPR− associated endonuclease. In some embodiments, the first CRISPR-associated endonuclease and the second CRISPR-associated endonuclease are orthogonal to each other, the first CRISPR− associated endonuclease recognizes the first and second copy of the second RNA cleavage site, and the second CRISPR-associated endonuclease recognizes the first and second copy of the first RNA cleavage site. In some embodiments, cleavage of the first copy and second copy of the first RNA cleavage site by the second RNA cleave effector leads to the expression of the second RNA cleavage effector and repression of the first RNA cleavage effector. In some embodiments, cleavage of the first copy and second copy of the second RNA cleavage site by the first RNA cleave effector leads to the expression of the first RNA cleavage effector and repression of the second RNA cleavage effector.

In some embodiments, the engineered bi-stable toggle switch comprises expression cassettes for a first and a second molecules. In some embodiments, the first expression cassette further comprises a coding sequence for a second output molecule operably joined to the coding sequence for the first RNA cleavage effector and a first spacer located between the coding sequence of the first RNA cleavage effector and the coding sequence for the second output molecule; and the second expression cassette further comprises a coding sequence for a first output molecule operably joined to the coding sequence for the second RNA cleavage effector and a second spacer located between the coding sequence of the second RNA cleavage effector and the coding sequence for the first output molecule. In some embodiments, the first and/or the second spacer is an internal ribosome entry site (IRES) or a 2A peptide (e.g., T2A or P2A). In some embodiments, the first and second output molecules are encoded on different constructs from the first and second expression cassettes. In some embodiments, the engineered bi-stable toggle switch further comprises a first output molecule expression cassette including, from 5′ to 3′, a promoter operably linked to: (i) optionally a nucleotide sequence encoding a third copy of the second RNA cleavage site, a first output molecule coding sequence, and optionally a nucleotide sequence encoding a third copy of the first RNA cleavage site; and a second output molecule expression cassette including, from 5′ to 3′, a promoter operably linked to: (i) optionally a nucleotide sequence encoding a third copy of the first RNA cleavage site, a second output molecule coding sequence, and optionally nucleotide sequence encoding a third copy of the second RNA cleavage site. In some embodiments, the first output molecule and the second out molecule are different.

An “output molecule,” as used herein, refers to a downstream molecule produced by the engineered bi-stable toggle switch. In some embodiments, when engineered bi-stable toggle switch is biased towards a first RNA cleavage effector high state (first high state), the expression of the second output molecule increases. In some embodiments, when engineered bi-stable toggle switch is biased towards a second RNA cleavage effector high state (second high state), the expression of the first output molecule increases. In some embodiments, the first output molecule has a basal expression level and the expression level increases (e.g., by at least 20% relative to the basal expression level) when the engineered bi-stable toggle switch is biased towards the second high state, compared to the first high state. In some embodiments, the second output molecule has a basal expression level and the expression level increases (e.g., by at least 20% relative to the basal expression level) when the engineered bi-stable toggle switch is biased towards the first high state, compared to the second high state. In some embodiments, the expression level of the first output molecule may be at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or higher relative to the basal expression level when in the second high state, compared to the first high state. In some embodiments, the expression level of the second output molecule may be at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or higher relative to the basal expression level when in the first high state, compared to the second high state.

The first and the second output molecule, in some embodiments, are detectable proteins. In some embodiments, a detectable protein is a fluorescent protein. A fluorescent protein is a protein that emits a fluorescent light when exposed to a light source at an appropriate wavelength (e.g., light in the blue or ultraviolet range). Suitable fluorescent proteins that may be used in accordance with the present disclosure include, without limitation, eYFP, mKO2, TagBFP, eGFP, eCFP, mKate2, mCherry, mPlum, mGrape2, mRaspberry, mGrape1, mStrawberry, mTangerine, mBanana, and mHoneydew. In some embodiments, a detectable protein is an enzyme that hydrolyzes a substrate to produce a detectable signal (e.g., a chemiluminescent signal). Such enzymes include, without limitation, beta-galactosidase (encoded by LacZ), horseradish peroxidase, or luciferase. In some embodiments, the output molecule is a fluorescent RNA. A fluorescent RNA is an RNA aptamer that emits a fluorescent light when bound to a fluorophore and exposed to a light source at an appropriate wavelength (e.g., light in the blue or ultraviolet range). Suitable fluorescent RNAs that may be used as an output molecule in the sensor circuit of the present disclosure include, without limitation, Spinach and Broccoli (e.g., as described in Paige et al., Science Vol. 333, Issue 6042, pp. 642-646, 2011).

In some embodiments, the first and the second output molecule are therapeutic molecules. A “therapeutic molecule” is a molecule that has therapeutic effects on a disease or condition, and may be used to treat a diseases or condition. Therapeutic molecules of the present disclosure may be nucleic acid-based or protein or polypeptide-based.

In some embodiments, nucleic acid-based therapeutic molecule may be an RNA interference (RNAi) molecule (e.g., a microRNA, siRNA, or shRNA) or an nucleic acid enzyme (e.g., a ribozyme). RNAi molecules and their use in silencing gene expression are familiar to those skilled in the art. In some embodiments, the RNAi molecule targets an oncogene. An oncogene is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). One skilled in the art is familiar with genes that may be targeted for the treatment of cancer.

Non-limiting examples of protein or polypeptide-based therapeutic molecules include enzymes, regulatory proteins (e.g., immuno-regulatory proteins), antigens, antibodies or antibody fragments, and structural proteins. In some embodiments, the protein or polypeptide-based therapeutic molecules are for cancer therapy.

Suitable enzymes (for operably linking to a synthetic promoter) for some embodiments of this disclosure include, for example, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase), esterases (e.g. sterol esterase), disaccharidases (e.g., sucrase, lactase, beta-galactosidase, maltase, isomaltase), DNases, and RNases.

Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma). In some embodiments, the antibody is an antibody that inhibits an immune check point protein, e.g., an anti-PD-1 antibody such as pembrolizumab (KEYTRUDA®) or nivolumab (OPDIVO®), or an anti-CTLA-4 antibody such as ipilimumab (YERVOY®). Other antibodies and antibody fragments may be operably linked to a synthetic promoter, as provided herein.

A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp1, and E2F1. Other transcription factors may be operably linked to a synthetic promoter, as provided herein.

As used herein, an immunoregulatory protein is a protein that regulates an immune response. Non-limiting examples of immunoregulatory include: antigens, adjuvants (e.g., flagellin, muramyl dipeptide), cytokines including interleukins (e.g., IL-2, IL-7, IL-15 or superagonist/mutant forms of these cytokines), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunoregulatory proteins may be operably linked to a synthetic promoter, as provided herein.

As used herein, an antigen is a molecule or part of a molecule that is bound by the antigen-binding site of an antibody. In some embodiments, an antigen is a molecule or moiety that, when administered to or expression in the cells of a subject, activates or increases the production of antibodies that specifically bind the antigen. Antigens of pathogens are well known to those of skill in the art and include, but are not limited to parts (coats, capsules, cell walls, flagella, fimbriae, and toxins) of bacteria, viruses, and other microorganisms. Examples of antigens that may be used in accordance with the disclosure include, without limitation, cancer antigens, self-antigens, microbial antigens, allergens and environmental antigens. Other antigens may be operably linked to a synthetic promoter, as provided herein.

In some embodiments, the antigen of the present disclosure is a cancer antigen. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-05. The cancer antigen may be selected from the group consisting of GAGE-1, GAGE-2, GAGE-3, GAGE-4, GAGE-5, GAGE-6, GAGE-7, GAGE-8 and GAGE-9. The cancer antigen may be selected from the group consisting of BAGE, RAGE, LAGE-1, NAG, GnT-V, MUM-1, CDK4, tyrosinase, p53, MUC family, HER2/neu, p21, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, PIA, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, CD20 and c-erbB-2. Other cancer antigens may be operably linked to a synthetic promoter, as provided herein.

In some embodiments, a protein or polypeptide-based therapeutic molecule is a fusion protein. A fusion protein is a protein comprising two heterologous proteins, protein domains, or protein fragments, that are covalently bound to each other, either directly or indirectly (e.g., via a linker), via a peptide bond. In some embodiments, a fusion protein is encoded by a nucleic acid comprising the coding region of a protein in frame with a coding region of an additional protein, without intervening stop codon, thus resulting in the translation of a single protein in which the proteins are fused together.

In some embodiments, the first and the second output molecules are functional molecules. A “functional molecule” refers to a molecule that is able to interact with other molecules or circuits to exert a function (e.g., transcription regulation, DNA or RNA cleavage, or any enzymatic activities). Exemplary functional molecules include, without limitation, enzymes (e.g., without limitation, nucleases), transcriptional regulators (e.g., without limitation, activators and repressors), RNAi molecules (e.g., without limitation, siRNA, miRNA, shRNA), and antibodies. In some embodiments, the functional molecule is a nuclease (e.g., a site-specific nuclease such as Csy4, Cas6, CasE, and Cse3). In some embodiments, the functional molecule is a transcriptional repressor (e.g., without limitation, TetR, CNOT7, DDX6, PPR10, and L7Ae). In some embodiments, having a functional molecule as the output molecule of the cleavage-induced transcript stabilizers described herein allows the cleavage-induced transcript stabilizer to further interact with downstream genetic circuits that contain elements responsive to the functional molecule produced by the cleavage-induced transcript stabilizer. Thus, “layering” of genetic circuits can be achieved, allowing multiple levels of complex regulation.

In some embodiments, the first expression cassette of the engineered bi-stable toggle switch further comprises a plurality of RNA degradation motifs at its 3′, and/or the second expression cassette of the engineered bi-stable toggle switch further comprises a plurality of RNA degradation motifs at its 3′. An “RNA degradation motif”, refers to a cis-acting nucleotide sequence that directs the RNA transcript to degradation, e.g., via the recruitment of enzymes involved in RNA degradation to the RNA molecule. Being “cis-acting” means that the RNA degradation motifs is part of the RNA transcript that it directs to degradation. In some embodiments, the degradation motifs are present in the 3′ untranslated region (3′UTR) or the RNA transcript. In some embodiments, the degradation motifs are appended at the 3′ end of the RNA transcript. In some embodiments, the first expression cassette and/or the second expression cassette of the engineered bi-stable toggle switch each comprises one or more RNA degradation motifs. In some embodiments, if the 3′ RNA degradation motifs on either the RNA transcript of the first expression cassette or the RNA transcript of the second expression cassette are not cleaved (e.g., cleavage happens at the RNA cleavage sites located at the 5′ end of the transcript), the RNA transcript would be rapidly degraded due to the presence of RNA degradation motifs in the RNA transcript. In some embodiments, if the 3′ RNA degradation motifs on either the RNA transcript of the first expression cassette or the RNA transcript of the second expression cassette are cleaved (e.g., cleavage happens at the RNA cleavage sites located at the 3′ end of the transcript, which removes the RNA degradation motifs), the RNA transcript would be stabilized.

In some embodiments, the first expression cassette and/or the second expression cassette of the engineered bi-stable toggle switch each comprises a plurality of RNA degradation motifs. In some embodiments, the first expression cassette and/or the second expression cassette of the engineered bi-stable toggle switch each comprises one or more RNA degradation motifs. In some embodiments, the first expression cassette and/or the second expression cassette of the engineered bi-stable toggle switch each comprises 1-50 repeats of the RNA degradation motifs. For example, the first expression cassette and/or the second expression cassette of the engineered bi-stable toggle switch each comprises 1-10, 1-20, 1-30, 1-40, 1-50, 10-50, 10-40, 10-30, 10-20, 20-50, 20-40, 20-30, 30-50, 30-40, or 40-50 repeats of the RNA degradation motifs. In some embodiments, the first expression cassette and/or the second expression cassette of the engineered bi-stable toggle switch each comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 RNA degradation motifs. In some embodiments, the first expression cassette and/or the second expression cassette of the engineered bi-stable toggle switch each comprises more than 50 (e.g., 60, 70, 80, 90, 100, or more) repeats of the RNA degradation motifs.

Non-limiting examples of RNA degradation motifs are sequences that recruit deadenylation complexes, miRNA target sites, aptamers that bind proteins associated with RNA degradation, or aptamers that bind engineered proteins that cause RNA degradation. In some embodiments, the RNA degradation motif is 5-30 nucleotides long. For example, the RNA degradation motifs may be 5-30, 5-25, 5-20, 5-15, 5-10, 10-30, 10-25, 10-20, 10-15, 15-30, 15-25, 15-20, 20-30, 20-25, or 25-30 nucleotides long. In some embodiments, the RNA degradation motifs is 5, 6, 7, 8, 9, 10, 11, 12, 13, 14, 15, 16, 17, 18, 19, 20, 21, 22, 23, 24, 25, 26, 27, 28, 29, or 30 nucleotides long. In some embodiments, longer (e.g., >30 nt) or shorter (e.g., <5 nt) RNA degradation motifs are used. In some embodiments, the RNA degradation motifs comprises a 8-nt RNA motif that naturally occurs in the 3′ UTR of human transcripts and directs the transcripts to degrade (e.g., as described in Geissler et al., Genes & Dev. 2016. 30: 1070-1085). Other known RNA degradation motifs that lead to degradation of RNA transcripts (e.g., as described in WO2019027869; Matoulkova et al., RNA Biology, 9:5, 563-576, 2012) may also be used in accordance with the present disclosure, including, without limitation: AU-rich elements, GU-rich elements, CA-rich elements, and introns.

In some embodiments, the presence of the RNA degradation motifs in the RNA transcript reduces the level and/or the half-life of the RNA transcript by at least 30%. For example, the presence of the RNA degradation motifs in the RNA transcript may reduce the level and/or the half-life of the RNA transcript by at least 30%, at least 40%, at least 50%, at least 100%, at least 3-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, or more. In some embodiments, the presence of the RNA degradation motifs in the RNA transcript reduces the level and/or the half-life of the RNA transcript by 30%, 40%, 50%, 100%, 3-fold, 5-fold, 10-fold, 20-fold, 30-fold, 40-fold, 50-fold, 60-fold, 70-fold, 80-fold, 90-fold, 100-fold, or more.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence. In some embodiments, the first promoter and the second promoter in the engineered bi-stable toggle switch are inducible promoters or constitutive promoter.

In some embodiments, a promoter is a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter.

In some embodiments, a promoter is an “inducible promoter,” which refer to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof. Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, the first expression cassette of the engineered bi-stable toggle switch further comprises a nucleotide sequence encoding a first transcript stabilization sequence located 3′ of the coding sequence for the first copy of the first RNA cleavage effector; and/or the second expression cassette of the engineered bi-stable toggle switch further comprises a nucleotide sequence encoding a first transcript stabilization sequence located 3′ of the coding sequence for the first copy of the first RNA cleavage effector. A “transcript stabilization sequence”, as used herein, refers to an RNA sequence that, when present in an RNA molecule (e.g., at the 5′ end or 3′ end), protects the RNA molecule from degradation. In some embodiments, the transcript stabilization sequence forms secondary structures that blocks access of exoribonucleases to the unprotected ends of the RNA molecule. The transcript stabilization sequence of the present disclosure is located between the RNA cleavage effector (e.g., CRISPR-associated endonuclease) coding sequence and the 3′ RNA cleavage site, and prevents degradation of the RNA cleavage effector (e.g., CRISPR-associated endonuclease) coding sequence. Non-limiting examples of RNA stabilizers that may be used in accordance with the present disclosure include: synthetic poly-adenylated tails, and stabilizing RNA triple helix structures (triplex) such as MALAT1 (e.g., as described in Brown et al., Nature Structural & Molecular Biology 21, 633-640, 2014), MEN0 triplex, KSHV PAN triplex, and histone stem loop. In some embodiments, the transcript stabilization sequence is a triplex. In some embodiments, the triplex is a Metastasis Associated Lung Adenocarcinoma Transcript 1 (MALAT1) triplex.

The transcript stabilization sequence stabilizes the RNA fragment containing nucleotide sequence encoding the RNA cleavage effectors and/or the output molecule, generated by cleavage of the RNA transcript by the RNA cleavage effector. An RNA fragment is considered to be stabilized when the half-life of the RNA fragment is at least 20% longer with of the RNA stabilizer, compared to without the RNA stabilizer. For example, an RNA fragment is considered to be stabilized when the half-life of the RNA fragment is increased by at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or more, compared to without the RNA stabilizer. In some embodiments, the half-life of the RNA fragment is increased by 20%, 30%, 40%, 50%, 60%, 70%, 80%, 90%, 100%, 2-fold, 5-fold, 10-fold, 50-fold, 100-fold or more, with the RNA stabilizer, compared to without the RNA stabilizer.

In some embodiments, the stabilizer further contributes to the stabilization of the RNA fragment containing nucleotide sequence encoding the output molecule, generated by cleavage of the RNA transcript by the RNA cleaver. In some embodiments, the half-life of the RNA transcript is increased by at least 30%, with the RNA stabilizer, compared to without the RNA stabilizer. For example, the half-life of the RNA transcript may be increased by at least 30%, at least 40%, at least 50%, at least 60%, at least 70%, at least 80%, at least 90%, at least 100%, at least 2-fold, at least 5-fold, at least 10-fold, at least 50-fold, at least 100-fold or more, with the RNA stabilizer, compared to without the RNA stabilizer. In some embodiments, the half-life of the RNA fragment is increased by 20%, 30%, 40%, 50%, 60%, 70%, 80%, 90%, 100%, 2-fold, 5-fold, 10-fold, 50-fold, 100-fold or more, with the RNA stabilizer, compared to without the RNA stabilizer.

In some embodiments, stabilization of the RNA transcript leads to increased expression of the output molecule. In some embodiments, the expression level of the output molecule is increased by at least 20%, when the degradation signal is cleaved, compared to before it was cleaved. For example, the expression level of the output molecule may be increased by at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or more, when the degradation signal is cleaved, compared to before it was cleaved. In some embodiments, the expression level of the output molecule is increased by 20%, 30%, 40%, 50%, 60%, 70%, 80%, 90%, 100%, 2-fold, 5-fold, 10-fold, 50-fold, 100-fold or more, when the degradation signal is cleaved, compared to before it was cleaved.

Also provided by the present disclosure, are additional elements to be incorporated into the engineered bi-stable toggle switch for control of the toggle switch behavior.

(i) Engineered Bi-Stable Toggle Switch with Protein Level Degradation Domain

The engineered bi-stable toggle switch of the present disclosure, can be modulated to be biased towards the first high state by adding the first RNA cleavage effector other than the amount of the first RNA cleavage effector produced by the first expression cassette; or the engineered bi-stable toggle switch can be modulated to be biased towards the second high state by adding the second RNA cleavage effector other than the amount of the second RNA cleavage effector produced by the second expression cassette. For the purpose of easily switching between the two states, the additional first RNA cleavage effector and the additional second RNA cleavage effector can be delivered to the cells already comprise the engineered bi-stable toggle switch when they are fused to protein degradation domains. A “protein degradation domain,” as used herein, refers to an amino acid sequence that induces the degradation of the protein/polypeptide it is fused to. In some embodiments, such protein degradation domains are responsive to small molecules. In some embodiments, in the absence of the cognate small molecule, the protein fused to the protein degradation domain is rapidly degraded. In some embodiments, in the presence of the cognate small molecule, the protein fused to the protein degradation domain is stable and can elicit its function. In some embodiments, adding the cognate small molecule of an RNA cleavage effector-protein degradation domain fusion protein can stabilize the fusion protein and enable the RNA cleavage effector to cleave the RNA cleavage sites, thus biasing the system to one state.

In some aspects, the engineered bi-stable toggle switch described herein further comprises: (iii) a third expression cassette comprising a third promoter operably linked to a coding sequence for a first fusion protein, wherein the first fusion protein comprises a second copy of the first RNA cleavage effector fused to a first protein degradation domain; and (iv) a fourth expression cassette comprising a fourth promoter operably linked to a coding sequence for a second fusion protein, wherein the second fusion protein comprises a second copy of the second RNA cleavage effector fused to a second protein degradation domain, wherein the third promoter and the fourth promoter are each constitutive promoters, wherein the first protein degradation domain is capable of binding to a first small molecule, wherein the second protein degradation domain is capable of binding to a second small molecule, and wherein the first small molecule and the second small molecule are different.

A fusion protein is a protein comprising two heterologous proteins, protein domains, or protein fragments, that are covalently bound to each other, either directly or indirectly (e.g., via a linker), or via a peptide bond. In some embodiments, a fusion protein is encoded by a nucleic acid comprising the coding region of a protein in frame with a coding region of an additional protein, without intervening stop codon, thus resulting in the translation of a single protein in which the proteins are fused together.

In some embodiments, the first fusion protein of the third expression cassette of the engineered bi-stable toggle switch is a fusion protein between the first RNA cleavage effector and the one or more first protein degradation domain, and/or the second fusion protein of the fourth expression cassette of the engineered bi-stable toggle switch is a fusion protein between the second RNA cleavage effector and the one or more second protein degradation domain. In some embodiments, the one or more first protein degradation domains are fused to the N-terminus of the first RNA cleavage effector; and/or the one or more second protein degradation domains are fused to the N-terminus of the second RNA cleavage effector.

In some embodiments, the protein degradation domains are sequences that recruit ubiquitin, recruit SUMO, trigger the unfolded protein response, bind protein degradation machinery, or increase the degradation rate of the protein by any other method. In some embodiments, the protein degradation domains are DDd, DDe or DDf. In some embodiments, the fusion proteins are any combination between one or more DDd, DDd, or DDf domains and the CRISPR-associated endonucleases. Non-limiting examples of the fusion proteins are DDe-Csy4, DDe-DDe-Csy4, DDd-Csy4, DDd-DDd-Csy4, DDe-CasE, DDe-DDe-CasE, DDd-CasE, DDd-DDd-CasE, DDe-Cas6, DDe-DDe-Cas6, DDd-Cas6, DDd-DDd-Cas6, DDe-Cse3, DDe-DDe-Cse3, DDd-Cse3, DDd-DDd-Cse3, DDe-LwaCas13a, DDe-DDe-LwaCas13a, DDd-LwaCas13a, DDd-DDd-LwaCas13a, DDe-PspCas13b, DDe-DDe-PspCas13b, DDd-PspCas13b, DDd-DDd-PspCas13b, DDe-RanCas13b, DDe-DDe-RanCas13b, DDd-RanCas13b, DDd-DDd-RanCas13b, DDe-PguCas13b, DDe-DDe-PguCas13b, DDd-PguCas13b, DDd-DDd-PguCas13b, DDe-RfxCas13d, DDe-DDe-RfxCas13d, DDd-RfxCas13d, or DDd-DDd-RfxCas13d. In some embodiments, the first fusion protein and the second fusion protein are DDe-Dde-Csy4 and DDd-CasE.

An exemplary nucleotide sequence
encoding the DDd domain is set
forth in SEQ ID NO: 3:
ATGATCAGTCTGATTGCGGCGTTAGCGGTAG
ATTACGTTATCGGCATGGAAAACGCCATGCC
GTGGAACCTGCCTGCCGATCTCGCCTGGTTT
AAACGCAACACCTTAAATAAACCCGTGATTA
TGGGCCGCCATACCTGGGAATCAATCGGTCG
TCCGTTGCCAGGACGCAAAAATATTATCCTC
AGCAGTCAACCGAGTACGGACGATCGCGTAA
CGTGGGTGAAGTCGGTGGATGAAGCCATCGC
GGCGTGTGGTGACGTACCAGAAATCATGGTG
ATTGGCGGCGGTCGCGTTATTGAACAGTTCT
TGCCAAAAGCGCAAAAACTGTATCTGACGCA
TATCGACGCAGAAGTGGAAGGCGACACCCAT
TTCCCGGATTACGAGCCGGATGACTGGGAAT
CGGTATTCAGCGAATTCCACGATGCTGATGC
GCAGAACTCTCACAGCTATTGCTTTGAGATT
CTGGAGCGGCGATGA
An exemplary nucleotide sequence
encoding the DDe domain is set
forth in SEQ ID NO: 4:
ATGAGCCTTGCCCTGTCACTTACAGCCGACC
AGATGGTTTCCGCGCTTCTCGACGCTGAACC
TCCAATTCTCTATTCCGAATACGACCCAACC
AGGCCGTTCTCCGAGGCATCTATGATGGGTC
TGCTGACAAATCTGGCAGACAGGGAACTGGT
GCACATGATCAATTGGGCGAAGCGCGTACCC
GGATTCGTCGATCTTGCACTCCATGATCAGG
TGCACTTGCTGGAGTGCGCTTGGATGGAGAT
CCTCATGATCGGGCTGGTGTGGCGGAGTATG
GAACACCCCGGCAAGTTGCTGTTTGCGCCTA
ACCTCCTGTTGGACAGGAACCAGGGGAAATG
TGTGGAGGGCGGTGTGGAAATCTTTGACATG
CTCCTCGCTACCTCAAGCCGGTTTAGGATGA
TGAATCTGCAGGGCGAAGAGTTCGTGTGTCT
CAAATCTATCATACTGTTGAACAGCGGAGTC
TACACCTTCCTCTCCAGTACTCTGAAATCTC
TGGAGGAGAAAGATCATATCCATCGCGTGCT
GGACAAGATAACCGACACGTTGATTCACTTG
ATGGCCAAAGCTGGGCTCACTCTGCAACAAC
AACATCAGCGACTGGCACAGCTGTTGCTGAT
TTTGAGCCACATTCGGCACATGTCCAGCAAG
AGAATGGAGCACCTCTATAGTATGAAGTGCA
AGAACGTCGTACCCCTGTCAGATCTGCTTCT
TGAAATGCTTGATGCCCACCGGTGA
An exemplary nucleotide sequence
encoding the DDe-DDe-Csy4 fusion
protein is set forth in
SEQ ID NO: 5:
ATGAGCCTTGCCCTGTCACTTACAGCCGACC
AGATGGTTTCCGCGCTTCTCGACGCTGAACC
TCCAATTCTCTATTCCGAATACGACCCAACC
AGGCCGTTCTCCGAGGCATCTATGATGGGTC
TGCTGACAAATCTGGCAGACAGGGAACTGGT
GCACATGATCAATTGGGCGAAGCGCGTACCC
GGATTCGTCGATCTTGCACTCCATGATCAGG
TGCACTTGCTGGAGTGCGCTTGGATGGAGAT
CCTCATGATCGGGCTGGTGTGGCGGAGTATG
GAACACCCCGGCAAGTTGCTGTTTGCGCCTA
ACCTCCTGTTGGACAGGAACCAGGGGAAATG
TGTGGAGGGCGGTGTGGAAATCTTTGACATG
CTCCTCGCTACCTCAAGCCGGTTTAGGATGA
TGAATCTGCAGGGCGAAGAGTTCGTGTGTCT
CAAATCTATCATACTGTTGAACAGCGGAGTC
TACACCTTCCTCTCCAGTACTCTGAAATCTC
TGGAGGAGAAAGATCATATCCATCGCGTGCT
GGACAAGATAACCGACACGTTGATTCACTTG
ATGGCCAAAGCTGGGCTCACTCTGCAACAAC
AACATCAGCGACTGGCACAGCTGTTGCTGAT
TTTGAGCCACATTCGGCACATGTCCAGCAAG
AGAATGGAGCACCTCTATAGTATGAAGTGCA
AGAACGTCGTACCCCTGTCAGATCTGCTTCT
TGAAATGCTTGATGCCCACCGGCTGATGAGC
CTTGCCCTGTCACTTACAGCCGACCAGATGG
TTTCCGCGCTTCTCGACGCTGAACCTCCAAT
TCTCTATTCCGAATACGACCCAACCAGGCCG
TTCTCCGAGGCATCTATGATGGGTCTGCTGA
CAAATCTGGCAGACAGGGAACTGGTGCACAT
GATCAATTGGGCGAAGCGCGTACCCGGATTC
GTCGATCTTGCACTCCATGATCAGGTGCACT
TGCTGGAGTGCGCTTGGATGGAGATCCTCAT
GATCGGGCTGGTGTGGCGGAGTATGGAACAC
CCCGGCAAGTTGCTGTTTGCGCCTAACCTCC
TGTTGGACAGGAACCAGGGGAAATGTGTGGA
GGGCGGTGTGGAAATCTTTGACATGCTCCTC
GCTACCTCAAGCCGGTTTAGGATGATGAATC
TGCAGGGCGAAGAGTTCGTGTGTCTCAAATC
TATCATACTGTTGAACAGCGGAGTCTACACC
TTCCTCTCCAGTACTCTGAAATCTCTGGAGG
AGAAAGATCATATCCATCGCGTGCTGGACAA
GATAACCGACACGTTGATTCACTTGATGGCC
AAAGCTGGGCTCACTCTGCAACAACAACATC
AGCGACTGGCACAGCTGTTGCTGATTTTGAG
CCACATTCGGCACATGTCCAGCAAGAGAATG
GAGCACCTCTATAGTATGAAGTGCAAGAACG
TCGTACCCCTGTCAGATCTGCTTCTTGAAAT
GCTTGATGCCCACCGGCTGATGGACCACTAT
CTCGACATTCGGCTGCGACCTGACCCGGAGT
TTCCTCCCGCCCAACTTATGAGCGTGCTGTT
CGGCAAATTGCACCAGGCCCTGGTAGCTCAA
GGCGGTGACCGAATTGGAGTGAGCTTCCCTG
ACCTGGATGAGTCTAGGTCCCGACTGGGTGA
GAGACTCAGAATCCACGCATCCGCCGACGAC
CTCAGAGCACTGCTGGCCCGCCCCTGGCTGG
AGGGCCTCAGAGATCACTTGCAGTTTGGAGA
GCCAGCCGTCGTGCCTCACCCTACCCCATAC
AGGCAAGTGTCTAGAGTCCAGGCCAAGAGTA
ACCCCGAACGGCTGCGGCGGAGGTTGATGAG
GCGGCACGACCTGTCCGAAGAAGAGGCACGG
AAAAGAATTCCCGACACCGTTGCTAGGGCTC
TTGATTTGCCCTTCGTCACCCTTCGATCACA
GTCCACCGGACAACATTTCCGCCTGTTCATT
AGGCACGGGCCTCTGCAGGTCACTGCCGAAG
AGGGCGGATTCACTTGCTACGGGCTGTCCAA
GGGAGGGTTCGTTCCATGGTTCTGA
An exemplary nucleotide sequence
encoding the DDd-CasE fusion
protein is set forth
in SEQ ID NO: 6:
ATGTACCTCAGTAAGATCATCATCGCCCGCG
CTTGGTCCCGTGACCTGTACCAACTGCACCA
AGAGCTCTGGCACCTCTTCCCCAACAGGCCA
GATGCCGCTAGAGACTTCCTGTTCCACGTGG
AGAAGCGTAACACCCCCGAAGGGTGCCACGT
GCTGTTGCAGAGTGCCCAGATGCCAGTGAGT
ACCGCTGTTGCCACTGTCATCAAGACTAAAC
AAGTTGAATTCCAACTGCAAGTGGGCGTCCC
TCTGTATTTCCGCCTCAGGGCCAACCCCATC
AAAACCATCCTGGACAACCAGAAGCGGCTGG
ATAGCAAAGGTAATATCAAGAGATGCCGCGT
GCCTCTGATCAAGGAGGCCGAGCAGATCGCT
TGGCTGCAACGCAAGCTGGGTAACGCCGCGA
GAGTGGAAGATGTGCACCCAATCTCCGAGCG
CCCGCAGTATTTCTCCGGGGAGGGGAAGAAC
GGCAAAATTCAGACTGTCTGCTTCGAGGGGG
TGCTCACTATTAACGACGCCCCTGCTCTGAT
CGACCTCCTGCAGCAGGGCATTGGGCCCGCG
AAGAGCATGGGATGCGGATTGTTGAGCCTGG
CACCCCTGATGATCAGTCTGATTGCGGCGTT
AGCGGTAGATTACGTTATCGGCATGGAAAAC
GCCATGCCGTGGAACCTGCCTGCCGATCTCG
CCTGGTTTAAACGCAACACCTTAAATAAACC
CGTGATTATGGGCCGCCATACCTGGGAATCA
ATCGGTCGTCCGTTGCCAGGACGCAAAAATA
TTATCCTCAGCAGTCAACCGAGTACGGACGA
TCGCGTAACGTGGGTGAAGTCGGTGGATGAA
GCCATCGCGGCGTGTGGTGACGTACCAGAAA
TCATGGTGATTGGCGGCGGTCGCGTTATTGA
ACAGTTCTTGCCAAAAGCGCAAAAACTGTAT
CTGACGCATATCGACGCAGAAGTGGAAGGCG
ACACCCATTTCCCGGATTACGAGCCGGATGA
CTGGGAATCGGTATTCAGCGAATTCCACGAT
GCTGATGCGCAGAACTCTCACAGCTATTGCT
TTGAGATTCTGGAGCGGCGATGA

As used herein, the term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain aspects, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain aspects, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible. In certain aspects, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. In certain aspects, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

In some embodiments, the small molecules capable of binding the protein degradation domains described herein are 4-hydroxytamoxifen (4-OHT) and trimethoprim (TMP). In some embodiments, DDe-DDe-Csy4 can be stabilized by small molecule 4-hydroxytamoxifen (4-OHT) and DDd-CasE can be stabilized by trimethoprim (TMP).

In addition, a converse design where protein degradation is enabled by binding of a small molecule to a protein degradation domain is also within the scope of present disclosure.

(ii) Engineered Bi-Stable Toggle Switch with Small Molecule Responsive Aptamer

Alternatively, the engineered bi-stable toggle switch of the present disclosure can be designed to incorporate small molecule-responsive aptamer sequence into the copies of the first and the second RNA cleavage sites. In some embodiments, binding of a small molecule to the aptamer within the RNA cleavage site induces a conformational change of the RNA cleavage hairpin, thus hindering the binding and cleavage of such RNA cleavage site by its cognate RNA cleavage effector (e.g., CRISPR-associated endonucleases).

In some embodiments, wherein the first and second copies of the first RNA cleavage site each comprises a first aptamer sequence capable of binding to a first small molecule, and binding of the first small molecule to the first RNA cleavage site is capable of blocking the second RNA cleavage effector from cleaving the first RNA cleavage site; wherein the first and second copies of the second RNA cleavage site each comprises a second aptamer sequence capable of binding to a second small molecule, and binding of the second small molecule to the second RNA cleavage site is capable of blocking the second RNA cleavage effector from cleaving the first RNA cleavage site, and wherein the first small molecule and the second small molecule are different.

In some embodiments, to further regulate the RNA degradation rate and/or the translation efficiency of the transcript from the first and the second expression cassettes in the engineered bi-stable toggle switch, an upstream open reading frame (upstream ORF) can be placed in the 5′UTR of each of the transcript. In some embodiments, the upstream open reading frame is a weak upstream ORF. In some embodiments, the upstream open reading frame is a strong upstream ORF. An exemplary nucleotide sequence encoding a weak upstream ORF is CTTATGGGTTGA (SEQ ID NO: 7). An exemplary nucleotide sequence encoding a strong upstream ORF is ACCATGGGTTGA (SEQ ID NO: 8)

In addition, a converse design where binding and cleavage of the RNA cleave site by its cognate RNA cleavage effector (e.g., CRISPR-associated endonucleases) is enabled by the conformational change induced by the binding of a small molecule to an aptamer sequence within the RNA cleavage site is also within the scope of present disclosure

(iii) Engineered Bi-Stable Toggle Switch with Ribozymes

Alternatively, the engineered bi-stable toggle switch of the present disclosure can be designed to incorporate RNA self-cleavage site at the 5′ of the first copy of the first RNA cleavage site, and the second copy of the second RNA cleavage site.

In some aspects, the first expression cassette of the engineered bi-stable toggle switch comprises a nucleotide sequence encoding a first RNA self-cleavage site operably linked to the first promoter, and wherein the nucleotide sequence encoding the first RNA self-cleavage site is located 5′ of the nucleotide sequence encoding the first copy of the first RNA cleavage site; and wherein the second expression cassette comprises a nucleotide sequence encoding a second RNA self-cleavage site operably linked to the second promoter, and wherein the nucleotide sequence encoding the second RNA self-cleavage site is located 5′ of the nucleotide sequence encoding the second copy of the second RNA cleavage site, wherein first RNA self-cleavage site is different from the second RNA self-cleavage site.

In some embodiments, the RNA-self cleavage sites are ribozymes. A “ribozyme” is an RNA molecule that is capable of catalyzing specific biochemical reactions, similar to the action of protein enzymes. In some embodiments, the ribozyme is a cis-acting ribozyme. A “cis-acting ribozyme” refers to a ribozyme that catalyzes self-cleavage (intramolecular or “in-cis” catalysis) from the RNA molecule that contains the ribozyme itself. In these instances, the cleavage site for the RNA cleaver in the RNA transcript of the present disclosure comprises the cis-acting ribozyme, which upon cleavage, excises itself and leaving two separated fragments of the RNA transcript. In some embodiments, the ribozyme is a trans-acting ribozyme. A “trans-acting ribozyme,” as used herein, refers to a ribozyme that cleaves an external substrate in a specific-manner. In these instances, the cleavage site for the RNA cleaver in the RNA transcript of the present disclosure comprises the recognition and cleavage sites for the trans-acting ribozyme. Suitable ribozymes that may be used in accordance with the present disclosure and their respective sequences include, without limitation: RNase P, hammerhead ribozymes, Hepatitis delta virus ribozymes, hairpin ribozymes, twister ribozymes, twister sister ribozymes, pistol ribozymes, hatchet ribozymes, glmS ribozymes, varkud satellite ribozymes, and spliceozyme. Naturally occurring ribozymes may be used. Further, ribozymes engineered such that they cleave their substrates in cis or in trans, e.g., as described in Carbonell et al. Nucleic Acids Res. 2011 March; 39(6): 2432-2444. Minimal ribozymes (i.e., the minimal sequence a ribozyme needs for its function, e.g., as described in Scott et al., Prog Mol Biol Transl Sci. 2013; 120: 1-23) may also be used in accordance with the present disclosure.

In some embodiments, the ribozymes are capable of self-cleavage in the absence of small molecules. In some embodiments, binding of a small molecule to the ribozyme induces self-cleavage of the ribozyme. In some embodiments, the first small molecule and the second small molecule are different.

In addition, a converse design where binding and ribozyme by its cognate small molecule inhibits self-cleavage of the ribozyme is also within the scope of present disclosure.

Also provided herein are the vectors comprising the engineered bi-stable toggle switch described herein. Each component of the engineered bi-stable toggle switch may be included in one or more (e.g., 2, 3 or more) nucleic acid molecules (e.g., vectors) and introduced into a cell. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press).

In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

In some embodiments, the engineered bi-stable toggle switch are is delivered to a cell by a vector. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000). A non-limiting example of a vector is a plasmid, RNA replicons, viral vectors (e.g., rAAV, lentivirus). Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector (e.g., retrovirus, adenovirus, adeno-associated virus, helper-dependent adenovirus systems, hybrid adenovirus systems, herpes simplex virus, pox virus, lentivirus, Epstein-Barr virus). In some embodiments, the viral vector is derived from an adeno-associated virus (AAV). In some embodiments, the viral vector is derived from an herpes simplex virus (HSV).

The nucleic acids or vectors containing the expression cassettes of the engineered bi-stable toggle switch may be delivered to a cell by any methods known in the art for delivering nucleic acids. For example, for delivering nucleic acids to a prokaryotic cell, the methods include, without limitation, transformation, transduction, conjugation, and electroporation. For delivering nucleic acids to a eukaryotic cell, methods include, without limitation, transfection, electroporation, and using viral vectors.

Also provided herein are the cells comprising the engineered bi-stable toggle switch or the vectors encoding the same as described herein. A “cell” is the basic structural and functional unit of all known independently living organisms. It is the smallest unit of life that is classified as a living thing. Some organisms, such as most bacteria, are unicellular (consist of a single cell). Other organisms, such as humans, are multicellular.

In some embodiments, a cell for use in accordance with the present disclosure is a prokaryotic cell, which may comprise a cell envelope and a cytoplasmic region that contains the cell genome (DNA) and ribosomes and various sorts of inclusions. In some embodiments, the cell is a bacterial cell. As used herein, the term “bacteria” encompasses all variants of bacteria, for example, prokaryotic organisms and cyanobacteria. Bacteria are small (typical linear dimensions of around 1 micron), non-compartmentalized, with circular DNA and ribosomes of 70S. The term bacteria also includes bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are gram-negative cells, and in some embodiments, the bacterial cells are gram-positive cells. Examples of bacterial cells that may be used in accordance with the invention include, without limitation, cells from Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Stremtomyces spp. In some embodiments, the bacterial cells are from Staphylococcus aureus, Bacillus subtilis, Clostridium butyricum, Brevibacterium lactofermentum, Streptococcus agalactiae, Lactococcus lactis, Leuconostoc lactis, Streptomyces, Actinobacillus actinobycetemcomitans, Bacteroides, cyanobacteria, Escherichia coli, Helobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus planta rum, Streptococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, Streptomyces ghanaenis, Halobacterium strain GRB, or Halobaferax sp. strain Aa2.2.

In some embodiments, a cell for use in accordance with the present disclosure is a eukaryotic cell, which comprises membrane-bound compartments in which specific metabolic activities take place, such as a nucleus. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including sperm, ova and embryonic cells, and non-reproductive cells, immune, kidney, lung, spleen, lymphoid, cardiac, gastric, intestinal, pancreatic, muscle, bone, neural, brain and epithelial cells. Stem cells, including embryonic stem cells or induced pluripotent stem cells, can also be used.

In some embodiments, the cell is a diseased cell. A “diseased cell,” as used herein, refers to a cell whose biological functionality is abnormal, compared to a non-diseased (normal) cell. In some embodiments, the diseased cell is a cancer cell.

In some embodiments, the cell is a cell used for recombinant protein production. Non-limiting examples of recombinant protein producing cells are Chinese hamster ovary (CHO) cells, human embryonic kidney (HEK)-293 cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

In some embodiments, the engineered bi-stable toggle switch is inserted into the genome of the cell. Methods of inserting genetic circuits into the genome of a cell are known to those skilled in the art (e.g., via site-specific recombination, using any of the known genome-editing tools, or using other recombinant DNA technology). In some instances, integrating the cleavage-induced transcript stabilizer into the genome of a cell is advantageous for its applications (e.g., therapeutic application or biomanufacturing application), compared to a cell engineered to simply express a transgene (e.g., via transcription regulation). It is known that genetically engineered cells suffer from epigenetic silencing of the integrated transgene. However, continuous transcription of transgenes helps to prevent their silencing, which is not possible with transcriptionally-regulated gene circuits relying on transcriptional repression. In contrast, the cleavage-induced transcript stabilizer described herein relies on RNA-level regulation and can achieve continuous transcription of the transgenes.

Also provided herein are animals comprising the engineered bi-stable toggle switch, the vectors encoding the same, or the cells comprising the engineered bi-stable toggle switch as described herein. In some embodiments, the non-human animal is a mammal. Non-limiting examples of non-human mammals are: mouse, rat, goat, cow, sheep, donkey, cat, dog, camel, or pig.

### II. Pharmaceutical Composition

In some aspects, the present disclosure, at least in part, relates to a pharmaceutical composition, comprising the engineered bi-stable toggle switch, the vector comprising the same, the cells, as described herein. The pharmaceutical composition described herein may further comprise a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used for in vivo administration must be sterile. This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In other embodiments, the pharmaceutical compositions described herein can be formulated for intra-muscular injection, intravenous injection, intratumoral injection or subcutaneous injection.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In other examples, the pharmaceutical composition described herein can be formulated in sustained-release format. Suitable examples of sustained-release preparations include semipermeable matrices of solid hydrophobic polymers containing the engineered bi-stable toggle switch, the vector comprising the same, or the cell comprising the same, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

Suitable surface-active agents include, in particular, non-ionic agents, such as polyoxyethylenesorbitans (e.g., TWEEN™ 20, 40, 60, 80 or 85) and other sorbitans (e.g., SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets having a suitable size and can have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### III. Applications

The present disclosure, at least in part, relates to the use of the engineered bi-stable toggle switch described herein.

In some embodiments, the present disclosure provides a method of switching gene expression between a first output molecule and a second output molecule, the method comprising: administering to a subject in need thereof the engineered bi-stable toggle switch of the vector, the cell, or the composition described herein.

In some embodiments, the present disclosure provides a method of maintaining long-term ON/OFF regulation of output molecule expression, the method comprising: administering to a subject in need thereof the engineered bi-stable toggle switch of the vector, the cell, or the composition described herein.

In some embodiments, the method described herein further comprises administering the subject with the first small molecule or the second small molecule. In some embodiments, the administration of the engineered bi-stable toggle switch is performed once in a lifetime, once every 10 years, once every 5 years, once every year, once every six month or once a month. In some embodiments, the administration of the small molecule to keep the engineered toggle switch at one state is performed more frequently compared to the engineered bi-stable toggle switch (e.g., once a month, once a week, once every other day, once a day, twice a day or more).

The engineered bi-stable toggle switch, the vector, the cells and the pharmaceutical composition described herein, can be used to treat various diseases (e.g., diseases treatable by the therapeutic molecules produced by the engineered bi-stable toggle switch).

To practice the method disclosed herein, an effective amount of any of the pharmaceutical compositions described herein can be administered to a subject (e.g., a human) in need of the treatment via a suitable route, such as intratumoral administration, by intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, pharmaceutical composition described herein can be aerosolized using a fluorocarbon formulation and a metered dose inhaler, or inhaled as a lyophilized and milled powder. In some examples, the pharmaceutical composition described herein is formulated for intratumoral injection. In particular examples, the pharmaceutical composition may be administered to a subject (e.g., a human patient) via a local route, for example, injected to a local site such as a tumor site or an infectious site.

As used herein, “an effective amount” refers to the amount of each active agent required to confer therapeutic effect on the subject, either alone or in combination with one or more other active agents. For example, the therapeutic effect can be reduced tumor burden, reduction of cancer cells, increased immune activity, reduction of a mutated protein, reduction of over-active immune response. Determination of whether an amount of engineered bi-stable toggle switch achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of pharmaceutical composition described herein may be appropriate. Various formulations and devices for achieving sustained release are known in the art.

In some embodiments, the treatment is a single injection of the pharmaceutical composition described herein. In some embodiments, the method described herein comprises administering to a subject in need of the treatment (e.g., a human patient) one or multiple doses of pharmaceutical composition described herein.

In some example, dosages for a pharmaceutical composition described herein may be determined empirically in individuals who have been given one or more administration(s) of the pharmaceutical composition. Individuals are given incremental dosages of the synthetic pharmaceutical composition described herein. To assess efficacy of the engineered bi-stable toggle switch, an indicator of the disease/disorder can be followed. For repeated administrations over several days or longer, depending on the condition, the treatment is sustained until a desired suppression of symptoms occurs or until sufficient therapeutic levels are achieved to alleviate a target disease or disorder, or a symptom thereof.

In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen of the pharmaceutical composition described herein used can vary over time.

For the purpose of the present disclosure, the appropriate dosage of the pharmaceutical composition described herein will depend on the specific miRNA signature of the cell and the miRNA to be expressed, the type and severity of the disease/disorder, the pharmaceutical composition described herein is administered for preventive or therapeutic purposes, previous therapy, the patient's clinical history and response to the engineered bi-stable toggle switch, and the discretion of the attending physician. A clinician may administer a pharmaceutical composition described herein, until a dosage is reached that achieves the desired result. Methods of determining whether a dosage resulted in the desired result would be evident to one of skill in the art. Administration of one or more pharmaceutical composition described herein can be continuous or intermittent, depending, for example, upon the recipient's physiological condition, whether the purpose of the administration is therapeutic or prophylactic, and other factors known to skilled practitioners.

The administration pharmaceutical composition described herein may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The subject to be treated by the methods described herein can be a mammal, such as a human, farm animals, sport animals, pets, primates, horses, dogs, cats, mice and rats. In one embodiment, the subject is a human.

In some embodiments, the subject may be a human patient having, suspected of having, or at risk for a disease. Non-limiting examples of diseases that are suitable for engineered bi-stable toggle switch based therapy are: Alpha-1 antitrypsin deficiency, Hypercholesterolemia, Hepatitis B infection, Liver adenoma due to HIV infection, Hepatitis C virus infection, Ornithine transcarbamylase deficiency, Hepatocellular carcinoma, Amyotrophic lateral sclerosis, Spinocerebellar ataxia type 1, Huntington's disease, Parkinson disease, Spinal and Bulbar muscular atrophy, Pyruvate dehydrogenase deficiency, Hyperplasia, obesity, facioscapulohumeral muscular dystrophy (FSHD), Nerve Injury-induced Neuropathic Pain, Age-related macular degeneration, Retinitis pigmentosa, heart failure, cardiomyopathy, cold-induced cardiovascular dysfunction, Asthma, Duchenne muscular dystrophy, infectious diseases, or cancer.

Non limiting examples of cancers include melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, gastric cancer, and various types of head and neck cancer, including squamous cell head and neck cancer. In some embodiments, the cancer can be melanoma, lung cancer, colorectal cancer, renal-cell cancer, urothelial carcinoma, or Hodgkin's lymphoma.

A subject having a target disease or disorder (e.g., cancer or an infectious disease) can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors associated with that disease/disorder. Such a subject can also be identified by routine medical practices.

In some embodiments, a pharmaceutical composition described herein may be co-used with another suitable therapeutic agent (e.g., an anti-cancer agent an anti-viral agent, or an anti-bacterial agent) and/or other agents that serve to enhance effect of a engineered bi-stable toggle switch. In such combined therapy, the pharmaceutical composition described herein, and the additional therapeutic agent (e.g., an anti-cancer therapeutic agent or others described herein) may be administered to a subject in need of the treatment in a sequential manner, i.e., each therapeutic agent is administered at a different time. Alternatively, these therapeutic agents, or at least two of the agents, are administered to the subject in a substantially simultaneous manner. Combination therapy can also embrace the administration of the agents described herein in further combination with other biologically active ingredients (e.g., a different anti-cancer agent) and non-drug therapies (e.g., surgery).

### IV. General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A laboratory notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Engineered bi-stable Toggle Switch

An important challenge in engineering synthetic genetic circuits in mammalian systems is epigenetic silencing. Transgene silencing has been observed in a large number of cell types including stem cells, neurons, CHO cells used for antibody production, and HEK293 cells. Epigenetic silencing has also been shown to be dependent on the transcriptional state of the gene, where strong constitutive expression may abrogate silencing. Since current genetic circuits often depend entirely on changing transcriptional activity, it is not surprising that such genetic circuits suffer from epigenetic silencing. Further, many genetic circuits are delivered as mRNAs. While RNA-based therapies are advantageous over DNA-based therapies, issues such as the fact that RNAbased circuits cannot be regulated by transcriptional controls remain. A post-transcriptional regulation platform could be designed to overcome these obstacles. Such a platform would allow integrated transgenes to be continuously expressed from verified constitutive promoters to combat epigenetic silencing, while regulation could be programmed post-transcriptionally.

As of yet, the field of synthetic biology has not produced a mammalian toggle switch that has shown good fold change between high and low states, stability of these states over multiple days, and responsiveness to switching events.

The engineered bi-stable toggle switch of the present disclosure enable cells to switch between stable states in response to an input from the user, while ensuring that these states support persistent and long-lasting cellular activity such as gene expression. These stable states include, but are not limited to, turning on and off production of a protein(s) of interest, and switching between production of different proteins of interest. These proteins of interest may be secreted or intracellular, and may have therapeutic efficacy, serve to change the behavior of the modified cell or other cells, or allow the study of the role of a gene of interest. Unlike a “drug-on” system that requires continuous user input, such as continuous delivery of a small molecule, the engineered bi-stable toggle switch of the present disclosure is capable of switching states in response to a transient input and then maintaining the new state in the absence of further inputs. Therefore, the toggle switch is particularly useful in a therapeutic context, where maintenance of a continuous drug concentration as an input to a drug-on system is logistically challenging, or a biomanufacturing context where maintenance of a continuous drug concentration can be prohibitively expensive.

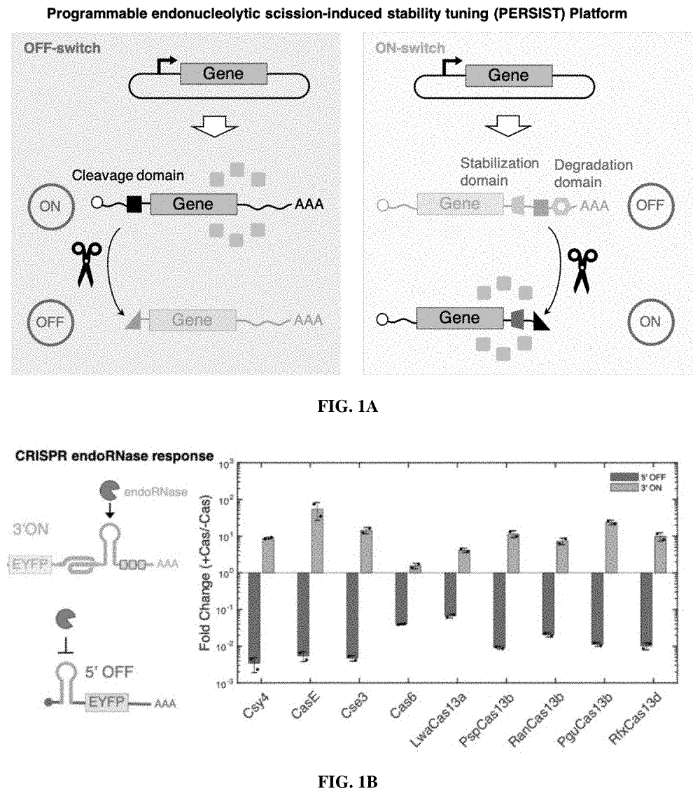

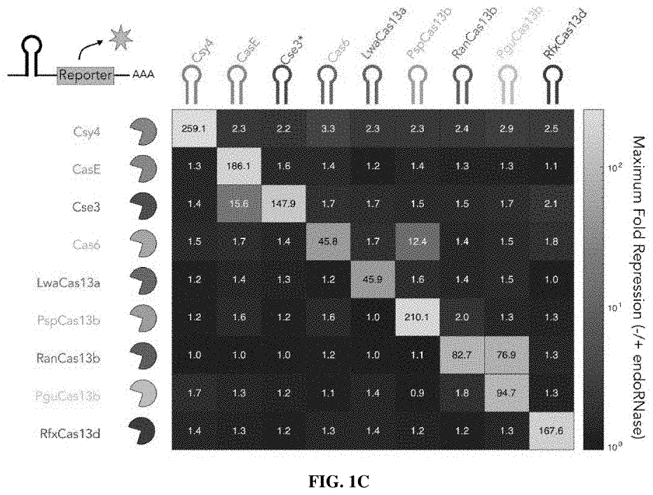

Despite the success of toggle switches in bacterial systems, construction of a toggle that is functional over long time scales is particularly challenging in mammalian cells. The only available toggle switches to date that have exhibited long-term bistability and switching in response to inputs have implemented the toggle via transcriptional regulation of the genes of interest, but mammalian cells tend to epigenetically silence genes synthetically regulated at the transcriptional level. For instance, synthetic transcription units that are inactive (e.g. the branch of the toggle that is in the OFF state), are notoriously difficult to (re)activate. The toggle approach presented herein is different in that it employs the RNA degradation-based Programmable Endonucleolytic Scission-Induced Stability Tuning (PERSIST) platform, allowing the creation of a toggle switch without transcriptional regulation where the components are constitutively expressed, and as such may be stable over very long time scales. The PERSIST system has been described in DiAndreth et al, PERSIST: A programmable RNA regulation platform using CRISPR endoRNases, bioRxiv preprint first posted online Dec. 16, 2019 (DiAndreth et al. bioRxiv. (2019). doi: 10.1101/2019.12.15.867150). The PERSIST platform provides for RNA cleavage events to act as either ON or OFF switches. These cleavage events can occur via RNAses, ribozymes, microRNAs, and other effectors of RNA cleavage. If a cleavage event occurs in the 5′ UTR of a transcript, the gene encoded in the transcript will lack 5′ transcript cap essential for translation and thus be repressed; if a cleavage event occurs in the 3′ UTR before a series of RNA degradation motifs and after a MALAT1 triplex structure, cleavage will stabilize the transcript and result in activation of the encoded gene (FIG. 1A-1B). The PERSIST platform includes the use of CRISPR endoRNAses in the Cas6 and Cas13b families to cleave RNAs containing recognition sequences of roughly 20 base pairs. Each of the 9 endoRNAses in the PERSIST platform is specific for its own recognition sequence, allowing parts to be composed into complex circuits.

First, RNA-level switches that enable turning transgene expression on or off through regulation of transcript degradation were designed. The RNA-level OFF-switch was designed such that the a transcript cleavage site was placed 5′ of the transgene, and the cleavage at this site, for example by miRNA or endoribonucleases (e.g., CRISPR endonucleases), can reduce transgene expression (FIG. 1A, left). Next, an RNA-level ON switch was designed to activate gene expression in response to transcript cleavage. Such RNA-level ON switch was designed to have three domains (FIG. 1A, right): (1) RNA degradation motifs that cause the rapid degradation of the transcript, (2) a cleavage domain which allows the removal of the degradation tag, and (3) a stabilizer that allows efficient translation and protects the mRNA after the removal of the RNA degradation motifs. Thus, the transcript is degraded in the absence of a cleavage event and stabilized post-cleavage. Each of the 9 endonucleases were tested for their ability to cleave their respective target sites as an RNA level ON and OFF switch. (FIG. 1B).

To demonstrate the utility of the platform, the orthogonality of different CRISPR endonuclease was evaluated. In particular, in order to be a platform that could be used in gene circuits, the endoRNase platform should have several characteristics: (1) the Cas proteins should be orthogonal to each other, i.e. have minimal cross-talk with each other's recognition sites, (2) the Cas protein-recognition site pairs should be modular such that recognition sites can be placed in either the 5′-OFF or 3′-ON PERSIST switch locations (in any order or combination) and have predictable behavior, and (3) the regulation enabled by the endoRNases should be composable, that is, it should be possible to connect endoRNases to create layered circuits.

The orthogonality of the endoRNAses were evaluated by testing each endoRNAse with every pairwise combination of Cas-responsive PERSIST-OFF reporters. Notably, CasE strongly cleaves the Cse3 recognition hairpin, but a single mutation U5A in the Cse3 recognition motif (Cse3*) renders it cleavable only by Cse3 and not CasE. As seen in FIG. 1C, the endoRNases' orthogonality suggests that these set of proteins are usable within the same circuit. Of note, some pairs should be avoided (unless beneficial for circuit design) such as RanCas13b:PguCas13b and CasE:Cse3 (with the wild type Cse3 recognition site). Given the large number of characterized Cas-family proteins with the ability to recognize and cleave specific RNA recognition motifs, PERSIST has the potential to expand beyond the nine proteins characterized here, making PERSIST scalable towards the construction of large and highly sophisticated genetic circuits.

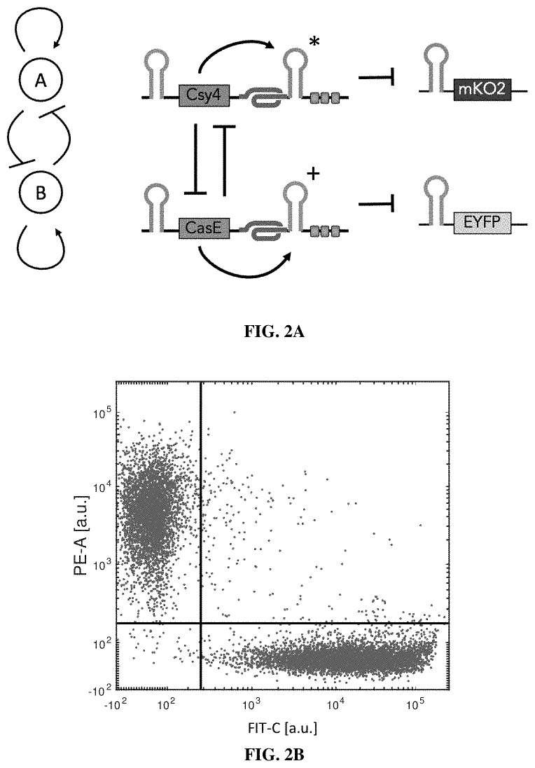

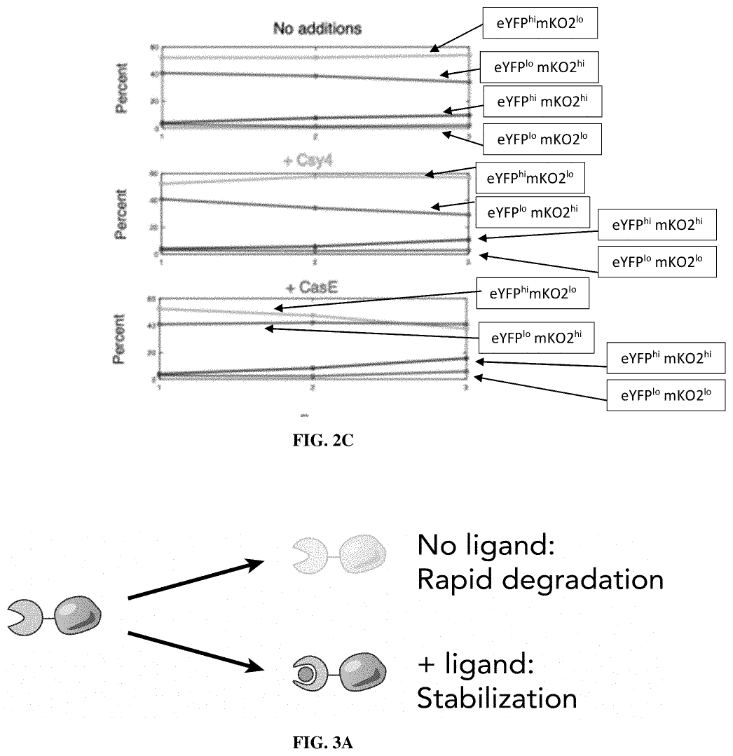

Accordingly, the PERSIST platform RNA-level ON and OFF switches were combined to configure the engineered bi-stable toggle switch of the present disclosure. Such an engineered bi-stable toggle switch includes two endoRNAses that repress each other and also activate themselves (FIGS. 2A-2C). Based on the evaluation of the endoRNAses, the pair of Csy4 and CasE were first selected to be tested in the engineered bi-stable toggle switch. The engineered bi-stable toggle switch was designed to have two expression cassettes: (i) Csy4-PEST, from 5′ to 3′, comprises a first promoter operably linked to a first copy of a CasE cleavage site, a coding sequence for Csy4, a first copy of Csy4 cleavage site, a MALT1 triplex, and a plurality of RNA degradation motifs; and (ii) CasE-PEST, from 5′ to 3′, comprises a second promoter operably linked to a second copy of Csy4 cleavage site, a coding sequence for CasE, a second copy of CasE cleavage site, a MALT1 triplex, and a plurality of RNA degradation motifs (FIG. 2A). While the MALT1 triplex increases RNA stability once the 3′ cleavage removes the plurality of degradation domains, it is not necessary to be present in the engineered bi-stable toggle switch. The state of the toggle was read out via two fluorescent proteins each repressed by one of the endoRNAses. In this case, the expression of Csy4 represses mKO2 expression due to the 5′ Csy4 cleavage site of the mKO2 coding sequence; and CasE represses eYFP expression due to the 5′ CasE cleavage site of the eYFP coding sequence. However, it is also within the scope of the currently disclosure that the output molecule can be expressed either under the same promoter of the toggle switch or under a different promoter.

Plasmids encoding Csy4 and CasE with the PERSIST motifs indicated above, along with fluorescent reporter plasmids bearing PERSIST repression motifs from each endoRNAse, were transfected into HEK293FT cells by polytransfection and analyzed 2 days after transfection. The bi-stable toggle switch was able to show bistability across a wide range of ratios that result from different cells receiving different copy number of the plasmids due to the transfections. The genetic circuit delivered to each cell essentially performs a weighted random decision to exhibit either the Csy4 high (eYFP high) or CasE high (mKO2 high) state.

Additional experiments were performed to show that the bi-stable toggle switch described above can be switched from one state to the other by the addition of Csy4 or CasE. As shown in FIG. 2C, toggle switch-transfected cells were transfected a day later with inducer endoRNases: either Csy4, CasE, or dummy plasmids and analyzed for two more days. Percentage of cells in each state (high-mKO2/low-eYFP, high-eYFP/low-mKO2, high-eYFP/high-mKO2, and low-eYFP/low-mKO2) were calculated. Inducer endoRNase transfection efficiency was not tracked with fluorescent proteins so values represent evaluation of all cells regardless of transfection state. Data indicates that a larger percentage of cells transfected with endoRNase show switching to the expected state compared to a toggle control sample where no inducer endoRNase was introduced.

### Example 2: Engineered Bi-Stable Toggle Switch with Protein-Level Degradation Domains

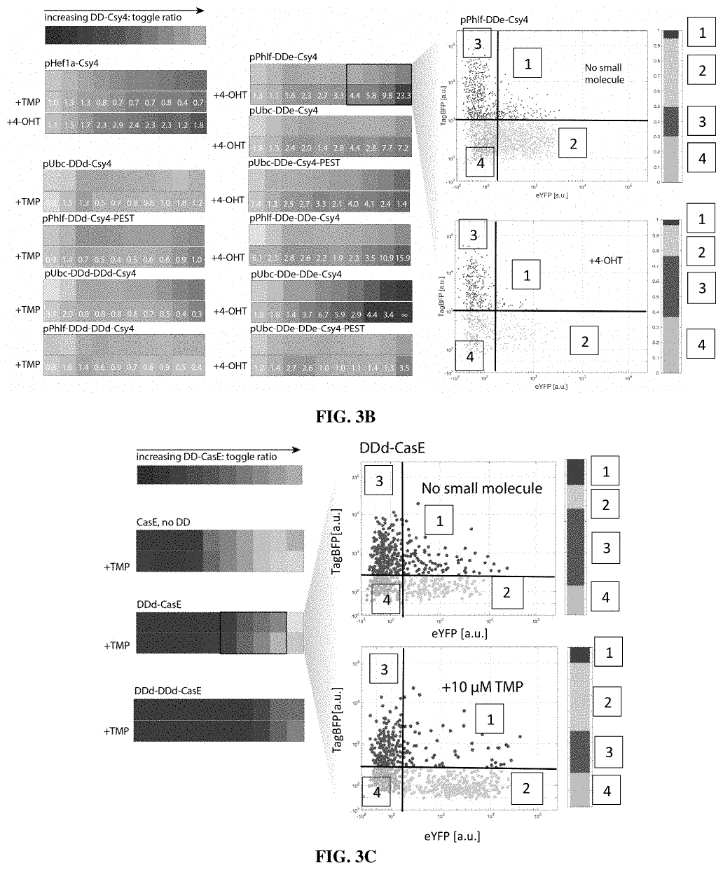

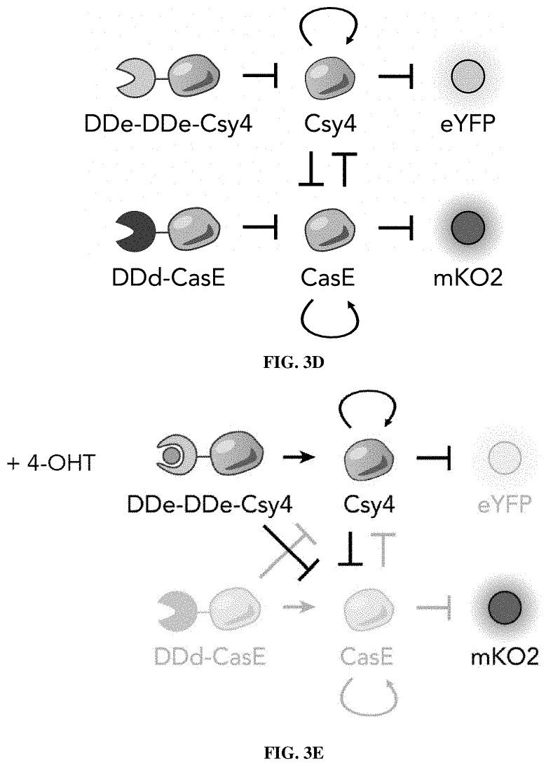

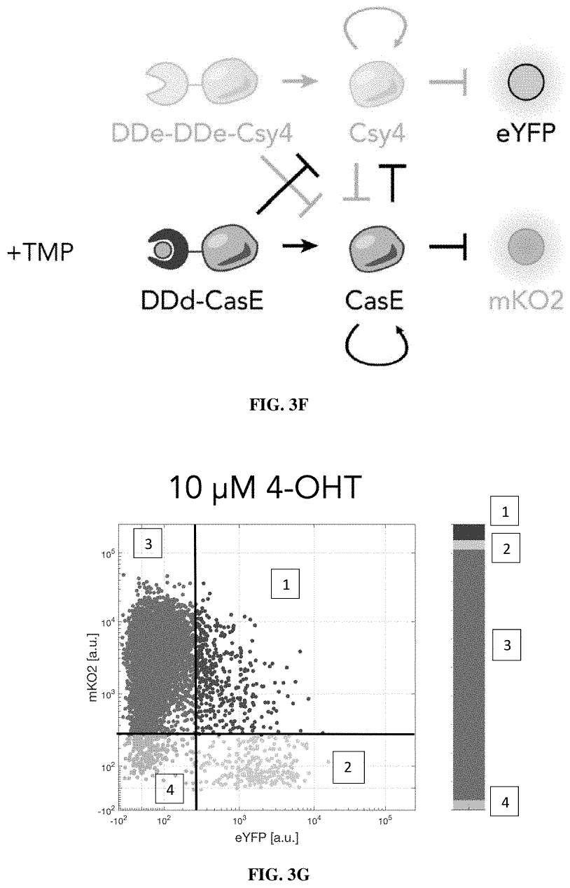

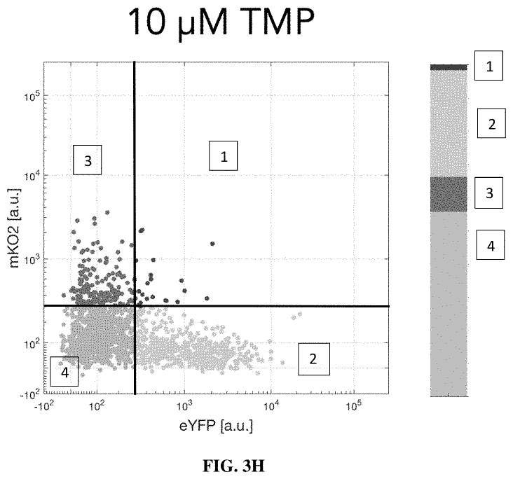

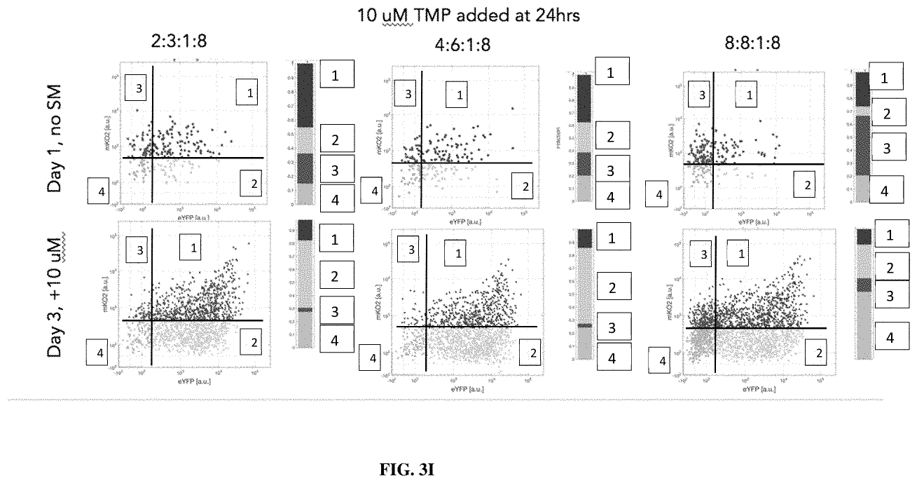

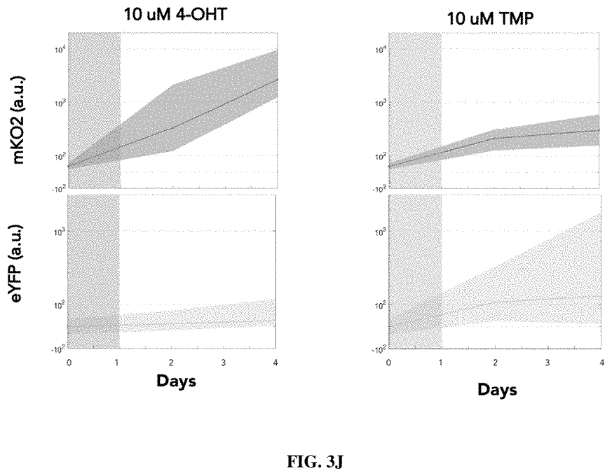

Additional elements can be incorporated into the basic engineered bi-stable toggle switch for long term stability at one state and for rapid switch between two different states. In one aspect of the disclosure, to achieve either a Csy4 high state or a CasE high state of the engineered bi-stable toggle switch described in Example 1, protein-level degradation domains that respond to small molecules were utilized. Various protein destabilization domains such as DDd, DDe, and DDf were tested. When such protein degradation domains were fused to a protein, the destabilization domain induces degradation of any protein it is fused to unless it is bound to a small molecule. In this design, the same engineered bi-stable toggle switch in Example 1 was used, with two additional transcriptional units. Each of the additional transcriptional units constitutively express one of the PERSIST RNAses fused to a different protein degradation domain, respectively. In the absence of a small molecule that can bind to the corresponding protein degradation domain, the fused PERSIST RNAse would be degraded; however, the introduction of the corresponding small molecule to the system would inhibit the degradation of the PERSIST RNAse. This design allowed the engineered bi-stable toggle switch to function as previously shown, but stabilize or switch state depending on the small molecule ligand added to the system (FIG. 3A). DDe and DDd were selected as the destabilization domains since they respond to FDA-approved small molecules: 4-hydroxytamoxifen (4-OHT) and trimethoprim (TMP), respectively. Nucleotide sequences encoding DDd and DDe domains are set forth in SEQ ID NO: 3 and SEQ ID NO: 4. DDd or DDe were fused to the N-terminus of the PERSIST RNAses.

The first step in developing this system was engineering fusion proteins between PERSIST RNAses and copies of DDds or DDes. All combinations of DDd, DDd-DDd, DDe, and DDe-DDe fused to the N terminus of Csy4, CasE, Cse3, PspCas13b, and RfxCas13d would be constructed and tested. Such fusion proteins would be degraded and lack RNA cleavage activity in the absence of small molecule; when the corresponding small molecule is present (e.g., 4-OHT for DDe and TMP for DDd), the fusion proteins would be stabilized such that they could cleave their respective target RNA site.

Csy4 was first selected to be fused with either one copy or two copies of DDd domain or DDe domain respectively: DDe-Csy4, DDe-DDe-Csy4, DDd-Csy4, and DDd-DDd-Csy4. Various promoters (e.g., phEF1α, pUbc, and pPh1f) were used to drive the expression of the Csy4 or Csy4-degradation domain fusion proteins. pPh1f promoters were tested in the presence of Gal4-NLS-VP64. As a result, the following constructs were generated: phEF1α-Csy4, pUbc-DDd-Csy4, pUbc-DDd-DDd-Csy4, pPh1f-DDd-DDd-Csy4, pPh1f-DDd-Csy4-PEST, pPh1f-DDe-Csy4, pUbc-DDe-Csy4, pUbc-DDe-Csy4-PEST, pPh1f-DDe-DDe-Csy4, pUbc-DDe-DDe-Csy4, and pUbc-DDe-DDe-Csy4-PEST. Each of the constructs and the engineered bi-stable toggle switch described in Example 1 were delivered to the cells by polytransfection (Gam et al, A ‘poly-transfection’ method for rapid, one-pot characterization and optimization of genetic systems, Nucleic Acids Research, Volume 47, Issue 18, 10 Oct. 2019, Page e106). The output molecules were eYFP and TagBFP. The system was designed such that Csy4 represses eYFP and CasE represses TagBFP. A cell in the CasE-high state expressed eYFP and a cell in the Csy4-high state expresses TagBFP. The ratio between CasE to Csy4 was set at 6.7 times as much CasE as Csy4, biasing the system towards a CasE-high state in the absence of additional proteins.

As indicated in FIG. 3B, the ratios between the engineered bi-stable motif and the DD-Csy4 fusion protein were achieved by binning polytransfections (polytransfection ratio shown in each row). As a control, phEF1α-Csy4 without DDs is able to switch the engineered bi-stable motif to a Csy4-high state without small molecule, with 4-OHT, or with TMP. Regardless of promoter, DDe-Csy4 and DDe-DDe-Csy4 show a higher fraction of cells in the TagBFP high state when 4-OHT is present than without, especially in higher ratio bins. For example, cells with between a 1.5:1 and a 30:1 ratio of DDe-Csy4 to engineered bi-stable motif are shown binned into TagBFP-high, eYFP-high, TagBFP and eYFP-high, or OFF states in the absence (top Row) and presence (bottom Row) of 4-OHT. As predicted eYFP high state dominates in the absence of 4-OHT, while TagBFP high state dominates in the presence of 4-OHT. In this Experiment, DDe-Csy4 and DDe-DDe-Csy4 showed the best capability of (i) being degraded in the absence of 4-OHT; and (ii) cleaving RNA efficiently in the presence of 4-OHT (FIG. 3B).

Further, fusion proteins between CasE and one or more DDd domains were screened similarly as described above. In this experiment, only hEF1α promoter was tested. Each of the construct and the engineered bi-stable toggle switch described in Example 1 were delivered to the cells by polytransfection. The output molecules were eYFP and TagBFP. The system was designed such that Csy4 represses eYFP and CasE represses TagBFP. A cell in the CasE-high state expressed eYFP and a cell in the Csy4-high state expresses TagBFP. The ratio between CasE to Csy4 was set at 1.6 times as much Csy4 as CasE, biasing the system towards a Csy4-high state in the absence of additional proteins. As a control, CasE without DDs is able to switch the engineered bi-stable toggle switch to a CasE-high state without or with TMP. As an example, cells with between a 1.5:1 and a 15:1 ratio of DDd-CasE to engineered bi-stable motif are shown binned into TagBFP-high, eYFP-high, TagBFP and eYFP high, or OFF states in the absence (top Row) and presence (bottom Row) of TMP. DDd-CasE was identified to be better at (i) being degraded in the absence of TMP; and (ii) cleaving RNA efficiently in the presence of TMP (FIG. 3C).

The pair of DDd-CasE and DDe-DDe-Csy4 were further tested for their ability to balance against each other in the absence of small molecules and to bias the engineered bi-stable toggle switch towards either a CasE or Csy4-high state, respectively, when 4-OHT or TMP was added. FIG. 3D showed schematic design of how DD-endoRNAse fusion proteins control the engineered bi-stable toggle switch. In the absence of the respective small molecules, DDe-DDe-Csy4 and DDd-CasE were produced but rapidly degrade, exerting minimal effects on the engineered bi-stable toggle switch. When 4-OHT is added, the DDe-DDe-Csy4 protein is stabilized, repressing CasE-PEST and eYFP, and activating Csy4-PEST. In this case, the engineered bi-stable toggle switch can be stabilized at a mKO2 high state (FIG. 3E). When TMP is added, the DDd-CasE protein is stabilized, repressing Csy4-PEST and mKO2, and activating CasE-PEST. Output fluorescent protein genes may also contain PERSIST activation domains to decrease expression in the OFF state. In this case, the engineered bi-stable toggle switch can be stabilized at a mKO2 high state (FIG. 3F). To test the system in FIG. 3D, CasE-PEST, Csy4-PEST, DDd-CasE and DDe-DDe-Csy4 were transiently transfected into HEK293 cells and cultured with either 10 μM 4-OHT (FIG. 3G) or 10 μM TMP (FIG. 3H) for 2 days. The cells were then analyzed by flow cytometry, and the data showed in the flow cytometry plots indicate the red and yellow fluorescence of each cell due to mKO2 and eYFP, respectively; bar graphs indicate the fraction of cells binned into mKO2-high (mKO2>150 a.u. & eYFP<150 a.u.), eYFP-high (mKO2<150 a.u. & eYFP>150 a.u.), both-high, and non-high bins. mKO2 exhibits a mean fold change of 7.7 and a median fold change of 16.2; eYFP shows a mean fold change of 3.5 and a median fold change of 6.1. Further, HEK293 cells were transfected with CasE-PEST, Csy4-PEST, DDd-CasE and DDe-DDe-Csy4 transiently and cultured in the absence of small molecule for 24 hours. Flow cytometry run at 24 hrs, prior to addition of a small molecule, indicated roughly equal levels of mKO2 and eYFP expression. Next, 10 μM TMP was added to the system at 24 hrs and maintained for 48 hrs. Flow cytometry at this time point indicates that the system now showed higher frequencies of eYFP expression than mKO2 expression (FIG. 3I). Moreover, the engineered bi-stable toggle switch was able to maintain either Csy4-high/CasE-low or Csy4-low/CasE-high state once the corresponding small molecule is removed from the system. As shown in FIG. 3J, adding 4-OHT into the system for 24 hours induced the bi-stable toggle switch to stabilize on a Csy4-high/CasE-low state (mKO2-high/eYFP-low). Interestingly, such state was maintained for at least 72 hours after 4-OHT was removed. Similarly, adding TMP into the system for 24 hours induced the bi-stable toggle switch to stabilize on a Csy4-low/CasE-high state (mKO2-low/eYFP-high), and such state was maintained for at least 72 hours after TMP was removed.

In addition, the expression levels of each component in this system can be modulated in order to achieve minimizing output protein in the off state while maximizing output protein in the on state. Such modulation can be performed in a transient transfection context, or a genomically integrated context. In a transiently transfected system, the amounts of each construct in the transfection mix can be manipulated; in a genomically integrated context, several methods can be applied. For example, the genes in the circuit could be integrated into the genome multiple times (e.g., to achieve gene A at a twofold higher expression level than gene B, the transcriptional unit encoding gene A would be integrated at twice as many locations as gene B). Alternatively, the genes used in the circuit could be placed under the control of inducible promoters responsive to an input signal (e.g., TetON or TetOFF promoters), and the degree of expression could be modulated by the amount of input signal added to the system. In addition, the genes in the circuit could be placed under the control of constitutive promoters of different strengths. Moreover, the transcriptional units in the circuit could include elements that modulate the RNA degradation rate of the different transcripts, such as the degradation domains used in PERSIST, or the translational efficiency of the transcripts, such as upstream open reading frames (uORFs) in the 5′ UTR. Single plasmid constructs with uORFs were generated such that each of the component in this system have an uORFs to modulate their expression levels.

### Example 3: Engineered Bi-Stable Toggle Switch with Small Molecule Responsive Aptamers

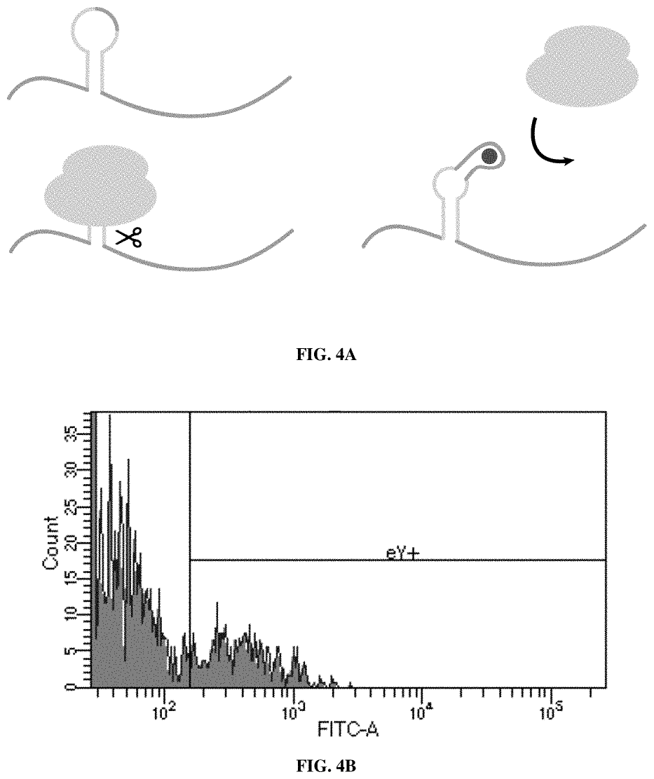

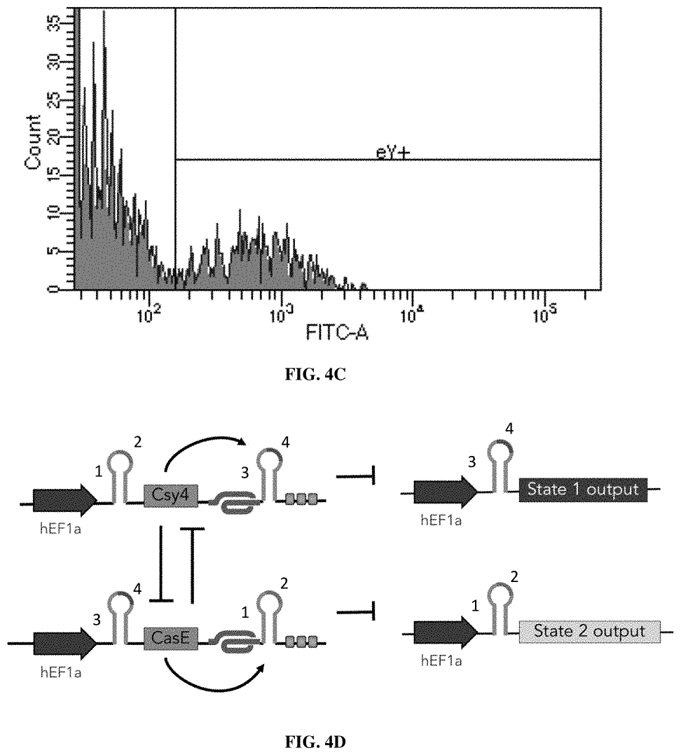

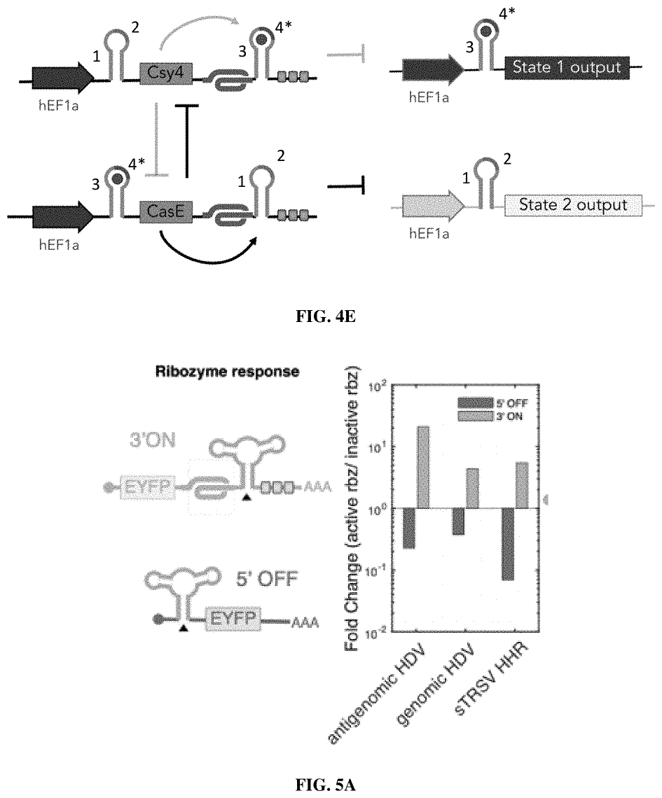

Alternatively, the engineered bi-stable toggle switch described in Example 1 can be designed to be able to respond to different small molecules to switch the system between different states. In this system, each of the endoRNAse target sites can include a small molecule-response aptamers. Binding of the small molecule to the aptamer stabilizes a secondary structure in the aptamer RNA that blocks the endoRNAse from cleaving its recognition hairpin. As illustrated by FIG. 4A, the CRISPR-endoRNAse recognition hairpin contains an aptamer sequence capable of binding to a small molecule. In the absence of the small molecule, the endoRNAse is able to cleave the recognition hairpin; however, the addition of the small molecule to the system induces a conformational change of the endoRNAse target hairpin, and such conformational change renders the endoRNAse incapable of binding and cleaving its target hairpin. A preliminary proof of concept test was performed to demonstrate the above concept. In this experiment, a CasE target hairpin containing a theophylline responsive aptamer was placed 5′ of an eYFP coding sequence in an eYFP-expressing construct. This eYFP-expressing construct was co-delivered to HEK293 cells with another construct expressing CasE. In the absence of theophylline, CasE was able to cleave the target hairpin located at the 5′ of the eYFP coding sequence, and repress its expression (FIG. 4B); however, in the presence of theophylline, the repression of CasE to eYFP was lifted, shown by the two-folds increase of eYFP expression (FIG. 4C).

Incorporation of such aptamer sequences into the endoRNAse target sites in the engineered bi-stable toggle switch would render the engineered bi-stable toggle switch capable of being controlled by small molecules. FIGS. 4D-4E show a schematic design of how Csy4's binding and cleavage of its target site (a target site having the aptamer sequence responsive to a small molecule) can be controlled by the absence (FIG. 4D) or presence (FIG. 4E) of the small molecule in an engineered bi-stable toggle switch.

Conversely to the current design, it is also within the scope of the current disclosure that a converse engineered bi-stable toggle switch can be designed such that the binding of a small molecule to an aptamer sequence in the endoRNAse target site can enable the binding and cleavage of such target site.

### Example 4: Engineered Bi-Stable Toggle Switch with Ribozymes

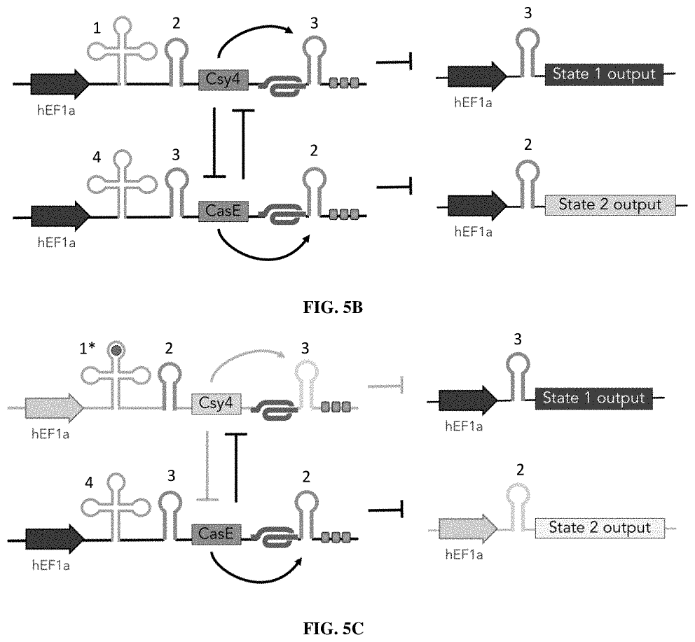

The engineered bi-stable toggle switch in Example 1 can also be designed to further incorporate ribozymes for the purpose of controlling the balance and biasing of the engineered bi-stable toggle switch. Proof of concept experiments were done to show that presence of a ribozyme either 5′ or 3′ of a eYFP coding sequence is able to repress or activate eYFP expression similarly to that of the PERSIST switch described in FIG. 1. As shown in FIG. 5A, PERSIST activation and repression of eYFP were successfully induced by the genomic sense direction of the hepatitis delta virus ribozyme (HDV), the antigenomic HDV ribozyme, and the hammerhead ribozyme (HHR).

The ribozymes capable of self-cleavage without a small molecule can be designed into the engineered bi-stable toggle switch. A first ribozyme (1) was placed 5′ to (upstream of) the first copy of the CasE target site (2),the Csy4 coding sequence, and Csy4 target site (3); and a second ribozyme (4) was placed 5′ to the second copy of the Csy4 target site (3), the CasE coding sequence, and the CasE target site (2). The first ribozyme (1) and the second ribozyme (2) are different ribozymes. The cleavage of the first ribozyme (1) represses the expression of Csy4, and the cleavage of the second ribozyme (4) represses the expression of CasE (FIG. 5B). Conversely, the ribozymes can be placed 3′ to (downstream of) the Csy4 or CasE coding sequence, such that the self-cleavage of the ribozymes would turn on the expression of Csy4 or CasE.

Alternatively, the ribozymes can be small molecule-responsive ribozymes such that the self-cleavage of the ribozymes can be induced by the addition of a small molecule. Such design is illustrated in FIG. 5C. In this system, the addition of the small molecule binding to the first ribozyme (1*) that is 5′ of the first copy of the CasE target site (2),Csy4 coding sequence, and Csy4 target site (3) would lead to repression of Csy4 such that the engineered bi-stable toggle switch can be biased to a CasE high state.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An engineered bi-stable toggle switch comprising:
(i) a first expression cassette comprising, from 5′ to 3′: a first promoter operably linked to a nucleotide sequence encoding a first copy of a first RNA cleavage site, a coding sequence for a first copy of a first RNA cleavage effector, a nucleotide sequence encoding a first copy of a second RNA cleavage site and a nucleotide sequence encoding a plurality of RNA degradation motifs; and
(ii) a second expression cassette comprising, from 5′ to 3′: a second promoter operably linked to a nucleotide sequence encoding a second copy of the second RNA cleavage site, a coding sequence for a first copy of a second RNA cleavage effector, a nucleotide sequence encoding a second copy of the first RNA cleavage site, and a nucleotide sequence encoding a plurality of RNA degradation motifs,
wherein the first RNA cleavage effector is orthogonal to the second RNA cleavage effector,
wherein the first RNA cleavage effector is capable of cleaving the second RNA cleavage site, and
wherein the second RNA cleavage effector is capable of cleaving the first RNA cleavage site.

2. The engineered bi-stable toggle switch of claim 1, wherein
the first expression cassette further comprises a nucleotide sequence encoding a first transcript stabilization sequence located 3′ of the coding sequence for the first copy of the first RNA cleavage effector; and/or
the second expression cassette further comprises a nucleotide sequence encoding a first transcript stabilization sequence located 3′ of the coding sequence for the first copy of the first RNA cleavage effector.

3. The engineered bi-stable toggle switch of claim 1, wherein
the first expression cassette further comprises a coding sequence for a second output molecule operably joined to the coding sequence for the first RNA cleavage effector and a first spacer located between the coding sequence of the first RNA cleavage effector and the coding sequence for the second output molecule; and
the second expression cassette further comprises a coding sequence for a first output molecule operably joined to the coding sequence for the second RNA cleavage effector and a second spacer located between the coding sequence of the second RNA cleavage effector and the coding sequence for the first output molecule, optionally wherein the first spacer and the second spacer is a nucleotide sequence encoding an internal ribosomal entry site (IRES) or a 2A peptide.

4. (canceled)

5. The engineered bi-stable toggle switch of claim 1, further comprising:
(iii) a third expression cassette comprising a third promoter operably linked to a coding sequence for a first fusion protein, wherein the first fusion protein comprises a second copy of the first RNA cleavage effector fused to a first protein degradation domain; and
(iv) a fourth expression cassette comprising a fourth promoter operably linked to a coding sequence for a second fusion protein, wherein the second fusion protein comprises a second copy of the second RNA cleavage effector fused to a second protein degradation domain,
wherein the third promoter and the fourth promoter are each constitutive promoters,
wherein the first protein degradation domain is capable of binding to a first small molecule,
wherein the second protein degradation domain is capable of binding to a second small molecule, and
wherein the first small molecule and the second small molecule are different.

6. The engineered bi-stable toggle switch of claim 5,
wherein the second copy of the first RNA cleavage effector is fused to the first protein degradation domain directly or through a linker; and/or
wherein the second copy of the second RNA cleavage effector is fused to the second protein degradation domain directly or through a linker;
optionally wherein the first fusion protein comprises more than one of the first protein degradation domain; and/or
optionally wherein the second fusion protein comprises more than one of the second protein degradation domain.

7. (canceled)

8. The engineered bi-stable toggle switch of claim 5,
wherein the first protein degradation domain is fused to the N-terminus of the first RNA cleavage effector, and/or
wherein the second protein degradation domain is fused to the N-terminus of the second RNA cleavage effector;
optionally wherein the first protein degradation domain and the second protein degradation domain are DDd, DDe, or DDf;
optionally wherein the first protein degradation domain is DDe and the first small molecule is 4-hydroxytamoxifen (4-OHT), and
the second protein degradation domain is DDd and the second small molecule is trimethoprim (TMP).

9-10. (canceled)

11. The engineered bi-stable toggle switch of claim 1,
wherein the first and second copies of the first RNA cleavage site each comprises a first aptamer sequence capable of binding to a first small molecule, and binding of the first small molecule to the first RNA cleavage site is capable of blocking the second RNA cleavage effector from cleaving the first RNA cleavage site,
wherein the first and second copies of the second RNA cleavage site each comprises a second aptamer sequence capable of binding to a second small molecule, and binding of the second small molecule to the second RNA cleavage site is capable of blocking the second RNA cleavage effector from cleaving the first RNA cleavage site, and
wherein the first small molecule and the second small molecule are different.

12. The engineered bi-stable toggle switch of claim 1,
wherein the first expression cassette comprises a nucleotide sequence encoding a first RNA self-cleavage site operably linked to the first promoter, and wherein the nucleotide sequence encoding the first RNA self-cleavage site is located 5′ of the nucleotide sequence encoding the first copy of the first RNA cleavage site; and
wherein the second expression cassette comprises a nucleotide sequence encoding a second RNA self-cleavage site operably linked to the second promoter, and wherein the nucleotide sequence encoding the second RNA self-cleavage site is located 5′ of the nucleotide sequence encoding the second copy of the second RNA cleavage site,
wherein first RNA self-cleavage site is different from the second RNA self-cleavage site.

13. The engineered bi-stable toggle switch of claim 12, wherein the first RNA self-cleavage site and the second RNA self-cleavage site are ribozymes,
optionally wherein the ribozymes are selected from the group consisting of antigenomic hepatitis delta virus (HDV) ribozyme, genomic HDV ribozyme, and sTRSV hammerhead ribozyme (HHR),
optionally wherein the first RNA self-cleavage site is capable of self-cleaving in response to a first small molecule,
optionally wherein the second RNA self-cleavage site is capable of self-cleaving in response to a second small molecule, and
optionally wherein the first small molecule and the second small molecule are different.

14-15. (canceled)

16. The engineered bi-stable toggle switch of claim 1, wherein the first promoter and the second promoter are constitutive promoters or inducible promoters.

17. The engineered bi-stable toggle switch of claim 2, wherein the first output molecule and the second output molecule are different, and wherein the first output molecule and the second output molecule are selected from the group consisting of: nucleic acids, therapeutic proteins, and detectable proteins.

18. The engineered bi-stable toggle switch of claim 1, wherein the first RNA cleavage effector and the second RNA cleavage effector are CRISPR endoribonucleases (endoRNAses), optionally wherein the CRISPR endoRNAses are Cas6, Csy4, CasE, Cse3, LwaCas13a, PspCas13b, RanCas13b, PguCas13b, or RfxCas13d.

19. (canceled)

20. The engineered bi-stable toggle switch of claim 2, wherein the first transcript stabilization sequence and the second transcript stabilization sequence each is a triplex, optionally wherein the triplex is a Metastasis Associated Lung Adenocarcinoma Transcript 1 (MALAT1) triplex.

21. (canceled)

22. The engineered bi-stable toggle switch of claim 1, wherein the plurality of RNA degradation motifs are RNA sequences capable of recruiting deadenylation complexes, miRNA target sites, aptamers comprising binding sites for proteins associated with RNA degradation, aptamers comprising binding sites for engineered proteins that cause RNA degradation.

23. A vector comprising the engineered bi-stable toggle switch of claim 1, optionally wherein the vector is a plasmid, an RNA replicon, or a viral vector, optionally wherein the viral vector is a lentiviral vector.

24-25. (canceled)

26. A cell comprising the engineered bi-stable toggle switch of claim 1,
optionally wherein the cell is a mammalian cell, optionally wherein the mammalian cell is a human induced pluripotent stem cell (hiPSC), a diseased cell, an immune cell, or a recombinant protein producing cell,
optionally wherein the cell comprises the engineered bi-stable toggle switch in its genome.

27-29. (canceled)

30. A non-human animal comprising the engineered bi-stable toggle switch of claim 1, optionally wherein the non-human animal is a mammal.

31. (canceled)

32. A composition comprising the engineered bi-stable toggle switch of claim 1 and optionally further comprising a pharmaceutically acceptable carrier.

33. (canceled)

34. A method of switching gene expression between a first output molecule and a second output molecule, or of maintaining long-term ON/OFF regulation of output molecule expression, the method comprising:
administering to a subject in need thereof the engineered bi-stable toggle switch of claim 1.

35. (canceled)

36. A method of switching gene expression between a first output molecule and a second output molecule, or of maintaining long-term ON/OFF regulation of output molecule expression, the method comprising administering to a subject in need thereof the engineered bi-stable toggle switch of claim 5, further comprising administering to the subject the first small molecule or the second small molecule.
